SEQUENCE LISTING (9 sequences)

SEQ ID NO: 1 - protein, 74 aa, Bacillus stearothermophilus
MYTNSDFVVIKALEDGVNVIGLTRGADTRFHHSEKLDKGEVLIAQFTEHTSAIKVRGKAYIQTRHGVIESEGKK

SEQ ID NO: 2 - DNA, 225 bp, Bacillus stearothermophilus
atgtatacgaacagcgactttgttgtcattaaagcgcttgaagacggagtgaacgtcattggattgacgcgcggggcggatacacggttccatcactcggaaaagctcgataaaggcgaagtgttgatcgcccagtttacagagcacacgtcggcgattaaagtgagaggcaaggcgtatattcaaacgcgccatggcgtcattgagtcggaagggaaaaagtaa

SEQ ID NO: 3 - DNA, 55 bp, artificial
  FW_XhoI_TRAP
ctgtacttccagagcggcggtagcggctcgagctacaccaactctgacttcgttg

SEQ ID NO: 4 - DNA, 37 bp, artificial
  RV_MluI_TRAP
ctcacgcgttattttttaccttcagattcgataacac

SEQ ID NO: 5 - DNA, 40 bp, artificial
  FW_BsrGI_tev
gctgtacaagctttctgaaaacctgtacttccagagcggc

SEQ ID NO: 6 - protein, 74 aa, artificial
  TRAP-K35C R64S
MYTNSDFVVIKALEDGVNVIGLTRGADTRFHHSECLDKGEVLIAQFTEHTSAIKVRGKAYIQTSHGVIESEGKK

SEQ ID NO: 7 - protein, 74 aa, artificial
  TRAP-K35C
MYTNSDFVVIKALEDGVNVIGLTRGADTRFHHSECLDKGEVLIAQFTEHTSAIKVRGKAYIQTRHGVIESEGKK

SEQ ID NO: 8 - protein, 335 aa, artificial
  H-mOrange-TRAP-K35C
MHHHHHHGGSSMVSKGEENNMAIIKEFMRFKVRMEGSVNGHEFEIEGEGEGRPYEGFQTAKLKVTKGGPLPFAWDILSPHFTYGSKAYVKHPADIPDYFKLSFPEGFKWERVMNYEDGGVVTVTQDSSLQDGEFIYKVKLRGTNFPSDGPVMQKKTMGWEASSERMYPEDGALKGKIKMRLKLKDGGHYTSEVKTTYKAKKPVQLPGAYIVDIKLDITSHNEDYTIVEQYERAEGRHSTGGMDELYKLSENLYFQSGGSGSSYTNSDFVVIKALEDGVNVIGLTRGADTRFHHSECLDKGEVLIAQFTEHTSAIKVRGKAYIQTRHGVIESEGKK

SEQ ID NO: 9 - protein, 335 aa, artificial
  H-mCherry-TRAP-K35C
MHHHHHHGGSSMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKLSENLYFQSGGSGSSYTNSDFVVIKALEDGVNVIGLTRGADTRFHHSECLDKGEVLIAQFTEHTSAIKVRGKAYIQTRHGVIESEGKK

(19) United States
(12) Patent Application Publication
(10) Pub. No.: US 2024/0122868 A1
(43) Pub. Date: Apr. 18, 2024

(54) AN ARTIFICIAL TRAP-CAGE, ITS USE AND METHOD OF PREPARING THEREOF

(71) Applicant: UNIWERSYTET JAGIELLONSKI, Krakow (PL)

(72) Inventors: Jonathan HEDDLE, Bishop Auckland (GB); Artur BIELA, Krakow (PL); Yusuke AZUMA, Krakow (PL); Izabela STUPKA, Krakow (PL); Karolina MAJSTERKIEWICZ, Wieliczka (PL)

(21) Appl. No.: 18/547,256

(22) PCT Filed: Feb. 24, 2022
(86) PCT No.: PCT/PL2022/050010

§ 371 Date: Aug. 21, 2023

(30) Foreign Application Priority Data
Feb. 24, 2021 (LU) LU102569
Feb. 24, 2021 (LU) LU102571
Feb. 24, 2021 (LU) LU102572
Feb. 24, 2021 (PL) P.437113
Feb. 24, 2021 (PL) P.437114
Feb. 24, 2021 (PL) P.437115

(51) Int. Cl. A61K 9/51; A61K 45/06; C07K 14/195

(52) U.S. Cl. CPC A61K 9/5169; A61K 9/5192; A61K 45/06; C07K 14/195

(57) ABSTRACT

The present invention provides an artificial TRAP-cage comprising a selected number of TRAP rings which are held in place by cross-linkers, wherein the cross-linkers are selected for their specific characteristics whereby the cages are programmable to be opened or remain closed on demand, under specific conditions.

### FIELD OF THE INVENTION

The present invention falls within the biochemistry field. It is related to an artificial protein cage called “TRAP-cage” comprising a selected number of TRAP rings which are held in place by molecular cross-linkers, wherein the cross-linkers are selected for their specific characteristics whereby the cages are programmable to be opened or remain closed on demand, under specific conditions.

### BACKGROUND

Protein complexes in nature represent important and highly sophisticated biological nanomachines and nano-structures. Large protein complexes in nature are typically constructed of a number of individual proteins held together by non-covalent interactions (i.e. hydrogen bonds, hydrophobic packing). This is particularly noticeable in protein cages such as capsids where multiple copies of identical protein subunits are held together in this way. In synthetic structural biology the ability to design and construct artificial protein assemblies may be useful, potentially allowing the introduction of properties and capabilities not present in nature. To this end new ways of connecting individual proteins together in defined ways is desirable.

Recently the inventors have studied such a possibility using TRAP (trp RNA-binding attenuation protein) from Geobacillus stearothermophilus as a nanometric building block. This TRAP adopts an oligomeric ring structure of 11 subunits in the native state and, along with a number of other ring proteins, has proven to be a useful bionano building block.

Having in mind disadvantages of known processes, the inventors have tried to find other methods for connecting protein subunits. Although there was some disclosure concerning binding two or other numbers of proteins via their cysteine SH groups, the inventors focused on this field taking into the consideration the use of gold as a “stitching” reagent.

The use of gold compounds to incorporate gold particles into nanostructures or providing nanoparticles as nanoclusters, protein cages for multiple applications, among others as a targeting molecule in delivery systems, is also well described in the literature as well as in patent documents and those ones are prior art for the present invention. For example, the International Application No PCT/KR2013/004454 describes a method for preparing a hyaluronic acid-gold nanoparticles/protein complex that can be used as a liver targeted drug delivery system, by surface modifying gold nanoparticles having excellent stability in the body with hyaluronic acid having biocompatibility, biodegradability and liver tissue-specific delivery properties, and binding protein drugs for treating liver diseases to the non-modified surface of the gold nanoparticles.

The U.S. Ser. No. 10/142,838 discloses the introduction of a precious metal atoms such as gold into a cage-like protein such as apoferritin by modifying the inner structure of a cage-like protein, and thus to form the precious metal—recombinant cage-like protein complex applicable to various microstructures.

The International Application No PCT/US2011/034190 discloses antibody-nanoparticle conjugates that include two or more nanoparticles (such as gold, palladium, platinum, silver, copper, nickel, cobalt, iridium, or an alloy of two or more thereof) directly linked to an antibody or fragment thereof through a metal-thiol bond.

Another example is U.S. Ser. No. 14/849,379 which discloses a recombinant self-assembled protein, comprising a target-oriented peptide fused to a self-assembled protein and a gold ion reducing peptide self-assembled.

The International Application No PCT/162018/056150 discloses a method for conjugation of free thiol group(s) containing biomolecules, leading to the biomolecular complex formation, comprising a reaction to connect biomolecules using a gold-donor agent in which a —S—Au—S— bond is formed, characterised in that a gold-donor agent is halogen(triarylphosphine)gold (I). Moreover, it has been there disclosed the use of halogen(triarylphosphine)gold (I) molecules as the gold-donor agent in the method of biomolecular complex formation.

The publication of A. D. Malay et al.: “An ultra-stable gold-coordinated protein cage displaying reversible assembly”; Nature 569 (2019): 438-442 (which is hereby incorporated by reference) discloses the TRAP-cage bonded together by single gold atoms, namely Au(I) ions which formed linear coordinate bonds between the thiol groups of pairs of cysteines.

In the present invention a new approach is realised—instead of by a —S—Au—S— bond-TRAP rings forming an artificial TRAP-cage are held in place by a cross-linker that is not made from metal atoms, cross-linkers are selected for their specific characteristics whereby the cages are programmable to be opened or remain closed on demand, under specific conditions. This approach allows control of the assembly and disassembly of the capsid-like protein complex, that is innovative in the view of the state of the art. A new approach regarding metal ions as cross-linkers is also demonstrated, also allowing allows control of the assembly and disassembly of the capsid-like protein complex, that is innovative in the view of the state of the art.

### SUMMARY OF THE INVENTION

The subject matter of the first aspect of the invention is an artificial TRAP-cage comprising a selected number of TRAP rings which are held in place by molecular cross-linkers, wherein the cross-linkers are molecules, and not single atoms, e.g. not metal atoms, selected for their specific characteristics whereby the cages are programmable to be opened or remain closed on demand, under specific conditions.

Preferably said specific conditions corresponds to the specific cleavage characteristic of the molecular cross-linker.

Using molecular cross-linkers, not single atomic cross-linkers, provides a greater degree of design choice and flexibility in designing the cages. These allow enhanced programmability, and control over the cross-linker cleavage characteristics. There is a much wider range of molecular cross-linkers available to choose from than (metallic) atomic cross-linkers.

All known TRAP-cage synthesis prior to this has utilised atomic gold or mercury cross-linking, no work contemplated the use of molecular cross-linkers. The larger size of molecules and the different chemistry required for them to cross-link the TRAP-rings means that it not obvious that molecular crosslinkers would be able to carry out the crosslinking function to result in an ordered cage formation, as all previous teaching focused on gold atoms.

Preferably the specific cleavage characteristic of the molecular cross-linker is selected from the group comprising:
(i) a reduction resistant/insensitive molecular cross-linker, whereby the cage remains closed under reducing conditions;
(ii) a reduction responsive/sensitive molecular cross-linker, whereby the cage opens under reducing conditions; and
(iii) a photoactivatable molecular cross-linker whereby the cage opens upon exposure to light.

Preferably the reduction resistant/insensitive molecular cross-linker can be selected from the group comprising: bismaleimideohexane (BMH), bisbromobimane and bis-bromoxylenes.

Preferably the reduction responsive/sensitive molecular cross-linker can be selected from the group comprising: dithiobismaleimideoethane (DTME).

Preferably the photoactivatable molecular cross-linker can be selected from the group comprising: bis-halomethyl benzene and its derivatives including 1,2-bis-bromomethyl-3-nitrobenzene (o-BBN), 2,4-bis-bromomethyl-1-nitrobenzene (m-BBN) and 1,3-bis-bromomethyl-4,6-dinitro-benzene (BDNB).

Preferably the molecular cross-linker is a homobisfunctional molecular moiety and its derivatives.

Preferably homobisfunctional molecular cross-linker is bismaleimideohexane (BMH).

Preferably the cage is resistant/insensitive to reducing conditions.

Preferably the homobisfunctional molecular cross-linker is dithiobismaleimideoethane (DTME).

Preferably the cage is responsive/sensitive to reducing conditions.

Preferably the molecular cross-linker is a bis-halomethyl benzene and its derivatives.

Preferably the molecular cross-linker is selected from the group comprising, 1,2-bis-bromomethyl-3-nitrobenzene (BBN), bis-bromoxylene and 1,3-bis-bromomethyl-4,6-dinitro-benzene (BDNB).

Preferably the molecular cross-linker is photolabile by exposure to UV light.

Preferably wherein the number of TRAP rings in the TRAP-cage is between 6 and 60, preferably between 7 and 55, preferably between 8 and 50, preferably between 9 and 45, preferably between 10 and 40, preferably between 11 and 35, preferably between 12 and 34, preferably between 13 and 33, preferably between 14 and 32, preferably between 15 and 31, preferably between 16 and 30, preferably between 17 and 29, preferably between 18 and 28, preferably between 19 and 27, preferably between 20 and 26. Preferably the number of TRAP rings in the TRAP-cage is less than 40, preferably less than 35, preferably less than 30. Preferably the number of TRAP rings in the TRAP-cage is more than 6, preferably more than 10, preferably more than 15, preferably more than 20.

Preferably, the number of TRAP rings in the TRAP-cage is between 12 and 24.

Preferably, the number of TRAP rings in the TRAP-cage is about 24, preferably 24.

Preferably the number of TRAP rings in the TRAP-cage is about 12, preferably 12.

Preferably the number of TRAP rings in the TRAP-cage is about 20, preferably 20.

Preferably, the cage according to the invention comprises a mixture of different programmable molecular cross-linkers.

Preferably, the cage according to the invention encapsulates a cargo that can be programmed to deliver said cargo in a specifically timed and desired location.

Preferably, the artificial TRAP-cage protein is modified to comprise any one or more of the following mutations selected from the group comprising K35C, R64S and K35C/R64S. Preferably the artificial TRAP-cage protein is modified to comprise a K35C mutation. Preferably, the cross-linker comprises dithiobismaleimideoethane (DTME) and preferably the artificial TRAP-cage protein is modified to comprise a K35C/R64S mutation.

Preferably, the cages according to the invention are hollow. Preferably the cage according to the invention is approximately spherical in shape, preferably a hollow sphere shape. Cages herein are hollow shapes roughly approximating a hollow sphere. These approximate the shape obtained when the TRAP rings are placed on the vertices or corners of regular convex polyhedra and then are linked together. In reference to any shape, vertices and edges are imaginary, i.e. there is not an actual physical polyhedron upon which the TRAP-rings are placed, rather the shape of the TRAP-cage is as if the rings are placed on the vertices or corners of regular convex polyhedral and then linked together.

Preferably, the TRAP-cages are stable in elevated temperatures, i.e. when the temperatures are elevated above normal room or human/animal body temperatures, preferably stable between 0 and 100° C., preferably stable between 15 and 100° C., preferably stable between 15 and 75° C., preferably stable up to 75° C., preferably stable at 75° C. and below.

Preferably, the TRAP-cages are stable in a non-neutral pH, preferably stable above pH 7 and below pH 7, preferably stable between pH 3 to 11, preferably stable between pH 4 to 10, preferably stable between pH 5 to 9.

Preferably, the TRAP-cages are stable in chaotropic agents (agents which disrupt hydrogen bonding in solution, which would disrupt or denature protein or macromolecular structures) or surfactants that would otherwise be expected to disrupt or denature protein or macromolecular structures. Preferably the cages show stability in n-butanol, ethanol, guanidinium chloride, lithium perchlorate, lithium acetate, magnesium chloride, phenol, 2-propanol, sodium dodecyl sulfate, thiourea, and urea. Preferably, the TRAP-cages are stable in up to 4 M GndHCl. Preferably, the TRAP-cages are stable in up to at least 7 M urea. Preferably, the TRAP-cages are stable in up to 15% of SDS. The stability of the cages described herein can be tested in standard conditions which would be known to the person of skill in the art using these agents to demonstrate said stability.

The cages described herein display unexpected stability in these conditions, providing more stable TRAP-cages than previously demonstrated.

The subject matter of the invention is also use of the cage according to the invention, as defined above, in delivery of a cargo in a controlled period and to a desired location.

The subject matter of the invention is also use of, any one or more of the group comprising a homobisfunctional molecular moiety and a bis-halomethyl benzene and its derivatives, as a programmable cross-linker in the construction of a programmable TRAP-cage.

The subject matter of the second aspect of the invention is also a method of preparing an artificial TRAP-cage, the method comprising:
(i) obtaining TRAP ring units by expression of the TRAP ring units in a suitable expression system and purification of the said units from the expression system;
(ii) conjugation of the TRAP ring units via at least one free thiol linkage with a programmable molecular cross-linker, wherein the cross-linker is selected for its specific characteristics;
(iii) formation of the TRAP-cage; and
(iv) purification and isolation of the TRAP-cages.

Preferably, step (ii) first comprises conjugation of the TRAP ring units via at least one metal cross-linker, preferably an atomic metal cross-linker. Step (ii) then comprises replacing the metal cross-linker with a molecular cross-linker. A molecular cross-linker may exchange metal atoms without changing orientation of the rings in the cage.

Preferably, the metal is gold. This altered step (ii) preferably applies when the cross-linker is photocleavable a linkers, preferably wherein the cross linker is bromoxylene or bisbromobimane.

Preferably programmable cross-linker is selected from the group comprising:
(i) a reduction resistant/insensitive linker, whereby the cage remains closed under reducing conditions;
(ii) a reduction responsive/sensitive linker, whereby the cage opens under reducing conditions; and
(iii) a photoactivatable linker whereby the cage opens upon exposure to light.

Preferably the reduction resistant/insensitive molecular cross-linker can be selected from the group comprising: bismaleimideohexane (BMH) and bis-bromoxylenes.

Preferably the reduction responsive/sensitive molecular cross-linker can be selected from the group comprising: dithiobismaleimideoethane (DTME).

Preferably the photoactivatable molecular cross-linker can be selected from the group comprising: bis-halomethyl benzene and its derivatives including 1,2-bis-bromomethyl-3-nitrobenzene (o-BBN), 2,4-bis-bromomethyl-1-nitrobenzene (m-BBN) and 1,3-bis-bromomethyl-4,6-dinitro-benzene (BDNB). Preferably, the artificial TRAP-cage protein is modified to comprise any one or more of the following mutations selected from the group comprising K35C, R64S and K35C/R64S. Preferably the artificial TRAP-cage protein is modified to comprise a K35C mutation.

Preferably, the cross-linker comprises dithiobismaleimideoethane (DTME) and preferably the artificial TRAP-cage protein is modified to comprise a K35C/R64S mutation.

Preferably wherein the number of TRAP rings in the TRAP-cage is between 6 and 60, preferably between 7 and 55, preferably between 8 and 50, preferably between 9 and 45, preferably between 10 and 40, preferably between 11 and 35, preferably between 12 and 34, preferably between 13 and 33, preferably between 14 and 32, preferably between 15 and 31, preferably between 16 and 30, preferably between 17 and 29, preferably between 18 and 28, preferably between 19 and 27, preferably between 20 and 26. Preferably the number of TRAP rings in the TRAP-cage is less than 40, preferably less than 35, preferably less than 30. Preferably the number of TRAP rings in the TRAP-cage is more than 6, preferably more than 10, preferably more than 15, preferably more than 20.

Preferably, the number of TRAP rings in the TRAP-cage is between 12 and 24.

Preferably, the number of TRAP rings in the TRAP-cage is about 24, preferably 24.

Preferably the number of TRAP rings in the TRAP-cage is about 12, preferably 12.

Preferably the number of TRAP rings in the TRAP-cage is about 20, preferably 20.

Preferably, step (ii) comprises conjugation with a mixture of different programmable cross-linkers.

Preferably, the reduction resistant/insensitive molecular cross-linker can be selected from the group comprising: bismaleimideohexane (BMH) and bis-bromoxylenes.

Preferably, the reduction responsive/sensitive molecular cross-linker can be selected from the group comprising: dithiobismaleimideoethane (DTME).

Preferably, the photoactivatable molecular cross-linker can be selected from the group comprising: bis-halomethyl benzene and its derivatives including 1,2-bis-bromomethyl-3-nitrobenzene (o-BBN), 2,4-bis-bromomethyl-1-nitrobenzene (m-BBN) and 1,3-bis-bromomethyl-4,6-dinitro-benzene (BDNB).

If no cysteine is present in the biomolecule, or they are present but not available for the reaction, —SH group, preferably as a group of cysteine, may be introduced into the biomolecule.

Introduction of cysteine can be carried out by any method known in the art. For example, but not limited to, the introduction of the cysteine is performed by methods known in the art, such as commercial gene synthesis or PCR-based site-directed mutagenesis using modified DNA primers. Above-mentioned methods are known by the persons skilled in the art and ready-to use kits with protocols are available commercially.

—SH moiety may be introduced into the biomolecule also by modification of other amino acids in the biomolecule i.e. by site-directed mutagenesis or by solid phase peptide synthesis.

The subject matter of the invention is also a TRAP-cage produced by this method. These cages may have any of the features or properties as described in relation to the first aspect of the invention, above, or anything else described herein.

The subject matter of the third aspect of the invention is an artificial TRAP-cage comprising a selected number of TRAP rings which are held in place by at least one cross-linker comprising a metal. Preferably the cross-linkers comprise only metal. Preferable the metal is a metal ion, preferably of a single type of metal.

Preferably, the metal cross-linker is selected for specific characteristics whereby the cages are programmable to be opened or remain closed on demand, under said specific conditions.

Preferably, the metal is selected from the group comprising Ag(I), Cd(II), Zn(II) and Co(II). The metals may be derivates of these metals.

Preferably, the metal is a d10 metal with a non-linear coordination geometry or shell. Preferably the d10 metal with a non-linear coordination geometry or shell is Zn(II) or Co(II).

Preferably, the metal is a d10 metal with a two-ligand linear coordination geometry or shell. Preferably the d10 metal with a non-linear coordination geometry or shell is Ag(I) or Cd(II).

Preferably, the artificial TRAP-cage protein is modified to comprise any one or more of the following mutations selected from the group comprising K35C, K35H, R64S, K35C/R64S, K35H/R64S, S33C, S33H, S33C/R64S, S33H/R64S, S33C/K35H S33H/K35H, S33C/K35C and S33H/K35C.

Preferably, the artificial TRAP-cage protein is modified to comprise a K35C/S33H mutation or a K35H/S33H mutation.

Preferably, the cross-linker comprises silver (Ag(I)) and preferably the artificial TRAP-cage protein is modified to comprise a K35C/R64S mutation.

Preferably, the cross-linker comprises cadmium (Cd(II)) and preferably the artificial TRAP-cage protein is modified to comprise a K35C/R64S mutation.

Preferably, the cross-linker comprises cobalt (Co(II)) and preferably the artificial TRAP-cage protein is modified to comprise a S33H/K35C or a S33H/K35H mutation.

Preferably, the cross-linker comprises zinc (Zn(II)) and preferably the artificial TRAP-cage protein is modified to comprise a S33H/K35C or a S33H/K35H mutation.

Preferably, wherein the number of TRAP rings in the TRAP-cage is between 6 and 60, preferably between 7 and 55, preferably between 8 and 50, preferably between 9 and 45, preferably between 10 and 40, preferably between 11 and 35, preferably between 12 and 34, preferably between 13 and 33, preferably between 14 and 32, preferably between 15 and 31, preferably between 16 and 30, preferably between 17 and 29, preferably between 18 and 28, preferably between 19 and 27, preferably between 20 and 26. Preferably the number of TRAP rings in the TRAP-cage is less than 40, preferably less than 35, preferably less than 30. Preferably the number of TRAP rings in the TRAP-cage is more than 6, preferably more than 10, preferably more than 15, preferably more than 20.

Preferably, the number of TRAP rings in the TRAP-cage is between 12 and 24.

Preferably, the number of TRAP rings in the TRAP-cage is about 24, preferably 24.

Preferably the number of TRAP rings in the TRAP-cage is about 12, preferably 12.

Preferably the number of TRAP rings in the TRAP-cage is about 20, preferably 20.

Preferably, the cage according to the invention comprises a mixture of different cross-linkers.

Preferably, the cage according to the invention encapsulates a cargo that can be programmed to deliver said cargo in a specifically timed and desired location.

Preferably the cages according to the invention are hollow. Preferably the cage according to the invention is approximately spherical in shape, preferably a hollow sphere. Cages herein are hollow shapes roughly approximating a hollow sphere. These approximate the shape obtained when the TRAP rings are placed on the vertices or corners of regular convex polyhedral and then linked together.

Preferably, the TRAP-cages are stable in elevated temperatures, i.e. when the temperatures are elevated above normal room or human/animal body temperatures, preferably stable between 0 and 100° C., preferably stable between 15 and 100° C., preferably stable between 15 and 75° C., preferably stable up to 75° C., preferably stable at 75° C. and below.

Preferably, the TRAP-cages are stable in a non-neutral pH, preferably stable above pH 7 and below pH 7, preferably stable between pH 3 to 11, preferably stable between pH 4 to 10, preferably stable between pH 5 to 9.

Preferably, the TRAP-cages are stable in chaotropic agents (agents which disrupt hydrogen bonding in solution, which would disrupt or denature protein or macromolecular structures) or surfactants that would otherwise be expected to disrupt or denature protein or macromolecular structures. Preferably the cages show stability in n-butanol, ethanol, guanidinium chloride, lithium perchlorate, lithium acetate, magnesium chloride, phenol, 2-propanol, sodium dodecyl sulfate, thiourea, and urea. Preferably, the TRAP-cages are stable in up to 4 M GndHCl. Preferably, the TRAP-cages are stable in up to at least 7 M urea. Preferably, the TRAP-cages are stable in up to 15% of SDS. The stability of the cages described herein can be tested in standard conditions which would be known to the person of skill in the art using these agents to demonstrate said stability.

The cages described herein display unexpected stability in these conditions, providing more stable TRAP-cages than previously demonstrated.

The subject matter of the invention is also use of the cage according to the invention, as defined above, in delivery of a cargo in a controlled period and to a desired location.

The subject matter of the invention is also use of, any one or more of the metals Ag(I), Cd(II), Zn(II) and Co(II) and their derivates as a cross-linker in the construction of a TRAP-cage.

The subject matter of the fourth aspect of the invention is also a method of preparing an artificial TRAP-cage, the method comprising:
(i) obtaining TRAP ring units by expression of the TRAP ring units in a suitable expression system and purification of the said units from the expression system;
(ii) conjugation of the TRAP ring units via at least one free thiol linkage with a metal cross-linker, wherein the cross-linker is selected for its specific characteristics;
(iii) formation of the TRAP-cage; and
(iv) purification and isolation of the TRAP-cages.

Preferably, the metal is selected from the group comprising Ag(I), Cd(II), Zn(II) and Co(II). The metals may be derivates of these metals.

Preferably the metal is a d10 metal with a non-linear coordination geometry or shell. Preferably the d10 metal with a non-linear coordination geometry or shell is Zn(II) or Co(II).

Preferably, the metal is a d10 metal with a two-ligand linear coordination geometry or shell. Preferably the d10 metal with a non-linear coordination geometry or shell is Ag(I) or Cd(II).

Preferably, the artificial TRAP-cage protein is modified to comprise any one or more of the following mutations selected from the group comprising K35C, K35H, R64S, K35C/R64S, K35H/R64S, S33C, S33H, S33C/R64S, S33H/R64S, S33C/K35H S33H/K35H, S33C/K35C and S33H/K35C.

Preferably, the artificial TRAP-cage protein is modified to comprise a K35C/S33H mutation or a K35H/S33H mutation.

Preferably, the cross-linker comprises silver (Ag(I)) and preferably the artificial TRAP-cage protein is modified to comprise a K35C/R64S mutation.

Preferably, the cross-linker comprises cadmium (Cd(II)) and preferably the artificial TRAP-cage protein is modified to comprise a K35C/R64S mutation.

Preferably, the cross-linker comprises cobalt (Co(II)) and preferably the artificial TRAP-cage protein is modified to comprise a S33H/K35C or a S33H/K35H mutation.

Preferably, the cross-linker comprises zinc (Zn(II)) and preferably the artificial TRAP-cage protein is modified to comprise a S33H/K35C or a S33H/K35H mutation.

Preferably wherein the number of TRAP rings in the TRAP-cage is between 6 and 60, preferably between 7 and 55, preferably between 8 and 50, preferably between 9 and 45, preferably between 10 and 40, preferably between 11 and 35, preferably between 12 and 34, preferably between 13 and 33, preferably between 14 and 32, preferably between 15 and 31, preferably between 16 and 30, preferably between 17 and 29, preferably between 18 and 28, preferably between 19 and 27, preferably between 20 and 26. Preferably the number of TRAP rings in the TRAP-cage is less than 40, preferably less than 35, preferably less than 30. Preferably the number of TRAP rings in the TRAP-cage is more than 6, preferably more than 10, preferably more than 15, preferably more than 20.

Preferably, the number of TRAP rings in the TRAP-cage is between 12 and 24.

Preferably, the number of TRAP rings in the TRAP-cage is about 24, preferably 24.

Preferably the number of TRAP rings in the TRAP-cage is about 12, preferably 12.

Preferably the number of TRAP rings in the TRAP-cage is about 20, preferably 20.

If no cysteine is present in the biomolecule, or they are present but not available for the reaction, —SH group, preferably as a group of cysteine, may be introduced into the biomolecule.

Introduction of cysteine can be carried out by any method known in the art. For example, but not limited to, the introduction of the cysteine is performed by methods known in the art, such as commercial gene synthesis or PCR-based site-directed mutagenesis using modified DNA primers. Above-mentioned methods are known by the persons skilled in the art and ready-to use kits with protocols are available commercially.

—SH moiety may be introduced into the biomolecule also by modification of other amino acids in the biomolecule i.e. by site-directed mutagenesis or by solid phase peptide synthesis.

Preferably, the method is carried out in part or in full in a HEPES buffer. Preferably the method is carried out at about pH 8.

The subject matter of the invention is also a TRAP-cage produced by this method. These cages may have any of the features or properties as described in relation to the first aspect of the invention, above, or anything else described herein.

The subject matter of the invention is also the use of any of the TRAP-cages described herein as a medicament.

The subject matter of the invention is also a method of treating a patient, comprising administering the TRAP-cages described herein to said patient.

The subject matter of the invention is also the use of any of the TRAP-cages described herein in treating a disease in a patient.

The subject matter of the invention is also a TRAP-cage produced by this method. These cages may have any of the features or properties as described in relation to the earlier aspects of the invention, above, or anything else described herein.

The subject matter of a further aspect of the invention is an artificial TRAP-cage comprising a selected number of TRAP rings which are held in place by at least one cross-linker. These cages may have any of the features or properties as described in relation to the earlier aspect of the invention, above, or anything else described herein.

Preferably the cross-linker is a metal. Preferably the cross-linkers comprise only metal. Preferable the metal is a metal ion, preferably of a single type of metal.

Preferably, the metal cross-linker is selected for specific characteristics whereby the cages are programmable to be opened or remain closed on demand, under said specific conditions.

Preferably, the metal is selected from the group comprising Au(I), Ag(I), Cd(II), Zn(II) and Co(II). The metals may be derivates of these metals.

Preferably, the metal is a d10 metal with a non-linear coordination geometry or shell. Preferably the d10 metal with a non-linear coordination geometry or shell is Zn(II) or Co(II).

Preferably, the metal is a d10 metal with a two-ligand linear coordination geometry or shell. Preferably the d10 metal with a non-linear coordination geometry or shell is Ag(I) or Cd(II).

Preferably, the artificial TRAP-cage protein is modified to comprise any one or more of the following mutations selected from the group comprising K35C, K35H, R64S, K35C/R64S, K35H/R64S, S33C, S33H, S33C/R64S, S33H/R64S, S33C/K35H S33H/K35H, S33C/K35C and S33H/K35C.

Preferably, the artificial TRAP-cage protein is modified to comprise a K35C/S33H mutation or a K35H/S33H mutation.

Preferably, the cross-linker comprises silver (Ag(I)) and preferably the artificial TRAP-cage protein is modified to comprise a K35C/R64S mutation.

Preferably, the cross-linker comprises cadmium (Cd(II)) and preferably the artificial TRAP-cage protein is modified to comprise a K35C/R64S mutation.

Preferably, the cross-linker comprises cobalt (Co(II)) and preferably the artificial TRAP-cage protein is modified to comprise a S33H/K35C or a S33H/K35H mutation.

Preferably, the cross-linker comprises zinc (Zn(II)) and preferably the artificial TRAP-cage protein is modified to comprise a S33H/K35C or a S33H/K35H mutation.

Preferably, the cross-linker comprises gold (Au(I)) and preferably the artificial TRAP-cage protein is modified to comprise a S33C/R64S.

Preferably the specific cleavage characteristic of the molecular cross-linker is selected from the group comprising: (i) a reduction resistant/insensitive molecular cross-linker, whereby the cage remains closed under reducing conditions; (ii) a reduction responsive/sensitive molecular cross-linker, whereby the cage opens under reducing conditions; and (iii) a photoactivatable molecular cross-linker whereby the cage opens upon exposure to light.

Preferably the reduction resistant/insensitive molecular cross-linker can be selected from the group comprising: bismaleimideohexane (BMH), bisbromobimane and bis-bromoxylenes.

Preferably the reduction responsive/sensitive molecular cross-linker can be selected from the group comprising: dithiobismaleimideoethane (DTME).

Preferably the photoactivatable molecular cross-linker can be selected from the group comprising: bis-halomethyl benzene and its derivatives including 1,2-bis-bromomethyl-3-nitrobenzene (o-BBN), 2,4-bis-bromomethyl-1-nitrobenzene (m-BBN) and 1,3-bis-bromomethyl-4,6-dinitro-benzene (BDNB).

Preferably the molecular cross-linker is a homobisfunctional molecular moiety and its derivatives. Preferably homobisfunctional molecular cross-linker is bismaleimideohexane (BMH).

Preferably the cage is resistant/insensitive to reducing conditions. Preferably the homobisfunctional molecular cross-linker is dithiobismaleimideoethane (DTME).

Preferably the cage is responsive/sensitive to reducing conditions. Preferably the molecular cross-linker is a bis-halomethyl benzene and its derivatives.

Preferably the molecular cross-linker is selected from the group comprising, 1,2-bis-bromomethyl-3-nitrobenzene (BBN), bis-bromoxylene and 1,3-bis-bromomethyl-4,6-dinitro-benzene (BDNB).

Preferably the molecular cross-linker is photolabile by exposure to UV light.

Preferably, the artificial TRAP-cage protein is modified to comprise any one or more of the following mutations selected from the group comprising K35C, R64S and K35C/R64S. Preferably the artificial TRAP-cage protein is modified to comprise a K35C mutation.

Preferably, the cross-linker comprises dithiobismaleimideoethane (DTME) and preferably the artificial TRAP-cage protein is modified to comprise a K35C/R64S mutation.

Preferably, wherein the number of TRAP rings in the TRAP-cage is between 6 and 60, preferably between 7 and 55, preferably between 8 and 50, preferably between 9 and 45, preferably between 10 and 40, preferably between 11 and 35, preferably between 12 and 34, preferably between 13 and 33, preferably between 14 and 32, preferably between 15 and 31, preferably between 16 and 30, preferably between 17 and 29, preferably between 18 and 28, preferably between 19 and 27, preferably between 20 and 26. Preferably the number of TRAP rings in the TRAP-cage is less than 40, preferably less than 35, preferably less than 30. Preferably the number of TRAP rings in the TRAP-cage is more than 6, preferably more than 10, preferably more than 15, preferably more than 20.

Preferably, the number of TRAP rings in the TRAP-cage is between 12 and 24.

Preferably, the number of TRAP rings in the TRAP-cage is about 24, preferably 24.

Preferably the number of TRAP rings in the TRAP-cage is about 12, preferably 12.

Preferably the number of TRAP rings in the TRAP-cage is about 20, preferably 20.

Preferably, the cage according to the invention comprises a mixture of different cross-linkers.

Preferably, the cage according to the invention encapsulates a cargo that can be delivered cargo in a specifically timed and desired location.

Preferably the cages according to the invention are hollow. Preferably the cage according to the invention is approximately spherical in shape, preferably a hollow sphere. Cages herein are hollow shapes roughly approximating a hollow sphere. These approximate the shape obtained when the TRAP rings are placed on the vertices or corners of regular convex polyhedral and then linked together.

Preferably, the TRAP-cages are stable in elevated temperatures, i.e. when the temperatures are elevated above normal room or human/animal body temperatures, preferably stable between 0 and 100° C., preferably stable between 15 and 100° C., preferably stable between 15 and 75° C., preferably stable up to 75° C., preferably stable at 75° C. and below.

Preferably, the TRAP-cages are stable in a non-neutral pH, preferably stable above pH 7 and below pH 7, preferably stable between pH 3 to 11, preferably stable between pH 4 to 10, preferably stable between pH 5 to 9.

Preferably, the TRAP-cages are stable in chaotropic agents (agents which disrupt hydrogen bonding in solution, which would disrupt or denature protein or macromolecular structures) or surfactants that would otherwise be expected to disrupt or denature protein or macromolecular structures. Preferably the cages show stability in n-butanol, ethanol, guanidinium chloride, lithium perchlorate, lithium acetate, magnesium chloride, phenol, 2-propanol, sodium dodecyl sulfate, thiourea, and urea. Preferably, the TRAP-cages are stable in up to 4 M GndHCl. Preferably, the TRAP-cages are stable in up to at least 7 M urea. Preferably, the TRAP-cages are stable in up to 15% of SDS. The stability of the cages described herein can be tested in standard conditions which would be known to the person of skill in the art using these agents to demonstrate said stability.

The cages described herein display unexpected stability in these conditions, providing more stable TRAP-cages than previously demonstrated.

The subject matter of the invention is also use of the cage according to the invention, as defined above, in delivery of a cargo in a controlled period and to a desired location.

The subject matter of the invention is also an TRAP-cage comprising a protein modified to comprise any one or more of the following mutations selected from the group comprising K35C, K35H, R64S, K35C/R64S, K35H/R64S, S33C, S33H, S33C/R64S, S33H/R64S, S33C/K35H S33H/K35H, S33C/K35C and S33H/K35C.

The subject matter of the invention is also aTRAP a protein modified to comprise any one or more of the following mutations selected from the group comprising K35C, K35H, R64S, K35C/R64S, K35H/R64S, S33C, S33H, S33C/R64S, S33H/R64S, S33C/K35H S33H/K35H, S33C/K35C and S33H/K35C.

### DETAILED DESCRIPTION OF THE INVENTION

Reference here to “TRAP protein” refers to Tryptophan RNA-binding attenuation protein, a bacterial protein. This protein can for example be isolated from wild type Geobacillus stearothermophilus, or other such bacteria. This protein can be isolated from various bacteria, but TRAP proteins which will work as described herein can be isolated from bacteria such as Alkalihalobacillus ligniniphilus, Anaerobacillus isosaccharinicus, Anoxybacillus caldiproteolyticus, Anoxybacillus calidus, Anoxybacillus pushchinoensis, Anoxybacillus tepidamans, Anoxybacillus tepidamans, Anoxybacillus vitaminiphilus, Bacillaceae bacterium, Bacillus alveayuensis, Bacillus alveayuensis, Bacillus sinesaloumensis, Bacillus sp. FJAT-14578, Bacillus sp. HD4P25, Bacillus sp. HMF5848, Bacillus sp. PS06, Bacillus sp. REN16, Bacillus sp. SA1-12, Bacillus sp. V3-13, Bacillus timonensis, Bacillus timonensis, Bacillus weihaiensis, Bacillus yapensis, Calidifontibacillus erzurumensis, Calidifontibacillus oryziterrae, Cytobacillus luteolus, Fredinandcohnia aciditolerans, Fredinandcohnia humi, Fredinandcohnia onubensis, Fredinandcohnia onubensis, Geobacillus genomosp. 3, Geobacillus sp. 46C-11a, Geobacillus stearothermophilus, Geobacillus stearothermophilus, Geobacillus stearothermophilus, Geobacillus stearothermophilus, Geobacillus stearothermophilus, Geobacillus stearothermophilus, Geobacillus stearothermophilus, Geobacillus thermodenitrificans NG80-2, Halobacillus dabanensis, Halobacillus halophilus, Halobacillus halophilus, Jeotgalibacillus proteolyticus, Litchfieldia alkalitelluris, Litchfieldia salsa, Mesobacillus harenae, Metabacillus, Metabacillus litoralis, Metabacillus sediminilitoris, Oceanobacillus limi, Oceanobacillus sp. Castelsardo, Ornithinibacillus, Ornithinibacillus bavariensis, Ornithinibacillus contaminans, Ornithinibacillus halophilus, Ornithinibacillus scapharcae, Parageobacillus caldoxylosilyticus, Parageobacillus genomo sp., Parageobacillus thermantarcticus, Parageobacillus thermantarcticus, Parageobacillus thermoglucosidasius, Parageobacillus thermoglucosidasius, Paucisalibacillus globulus, Paucisalibacillus sp. EB02, Priestia abyssalis, Priestia endophytica, Priestia filamentosa, Priestia koreensis, Priestia megaterium, Psychrobacillus glaciei, Salinibacillus xinjiangensis, Sutcliffiella cohnii, Thermolongibacillus altinsuensis. Trp RNA-binding attenuation protein is a bacterial, ring-shaped homo 11-mer (see A. A. Antson, J. Otridge, A. M. Brzozowski, E. J. Dodson, G. G. Dodson, K. S. Wilson, T. Smith, M. Yang, T. Kurecki, P. Gollnick, which is hereby incorporated by reference), The structure of trp RNA-binding attenuation, protein can be seen in the literature (Nature 374, 693-700 (1995), which is hereby incorporated by reference).

Suitably, the protein used herein is a modified version of wild-type TRAP isolated from Bacillus stearothermophilus. This is seen in Table 1:

TABLE 1

| Name | Protein sequence |
|---|---|
| Wild-type TRAP | MYTNSDFVVIKALEDGVNVIGLTRGADT |
| Bacillus | RFHHSEKLDKGEVLIAQFTEHTSAIKVR |
| stearothermophilus | GKAYIQTRHGVIESEGKK* |
| (PDB:1QAW) | [SEQ ID NO: 1] |

The Wild-type TRAP Bacillus stearothermophilus gene sequence is seen in Table 2:

TABLE 2

| | | Gene ID |
|---|---|---|
| Name | Gene sequence | (from UniProt) |
| Wild-type TRAP | Atgtatacgaacagcgactttgttgtcattaaag | 58572467 |
| Bacillus | cgcttgaagacggagtgaacgtcattggattg | |
| stearothermophilus | acgcgcggggcggatacacggttccatcact | |
| | cggaaaagctcgataaaggcgaagtgttgat | |
| | cgcccagtttacagagcacacgtcggcgatta | |
| | aagtgagaggcaaggcgtatattcaaacgcg | |
| | ccatggcgtcattgagtcggaagggaaaaag | |
| | taa | |
| | [SEQ ID NO: 2] | |

Preferably, preparation of proteins is performed by biomolecule expression in a suitable expression system and purification of the expression product. Preferably with a modified version of the above Wild-type TRAP Bacillus stearothermophilus gene sequence.

TRAP proteins forms rings, herein “TRAP rings”, and rings are the natural state of TRAP proteins. Typically, as is the case for the Geobacillus Stearothermophilus proteins as demonstrated herein, TRAP monomer proteins spontaneously assemble into toroids or rings made from monomers.

TRAP-cages only form under particular conditions, for example as demonstrated herein with the presence of cysteines that can be crosslinked resulting in rings assembling into a cage. For example as demonstrated herein, these will form with the presence of cysteine at position 35 (the result of a K35C mutation).

Reference herein to “TRAP ring” is synonymous with a TRAP building block, a subunit of the TRAP-cage complex or a TRAP monomer assembly. Reference herein to an “analog” of a particular protein or nucleotide sequence refers to a protein or nucleotide sequence having sufficient identity or homology to the protein or nucleotide sequence to be able to carry out the specified function, e.g. TRAP-cage formation under the conditions described herein, or encode a protein which is able to carry out the specified function, e.g. TRAP-cage formation under the conditions described herein.

To determine the percent identity/homology of two sequences, the sequence in question and a reference are aligned for optimal comparison purposes (e.g., gaps can be introduced in one or both of a first and a second amino acid or nucleic acid sequence for optimal alignment and non-homologous sequences can be disregarded for comparison purposes). A sequence may be determined an analog of a particular when it has preferably at least 40%, more preferably at least 50%, even more preferably at least 60%, and even more preferably at least 70%, 75%, 80%, 82%, 84%, 85%, 86%, 87%, 88%, 89%, 90%, 91%, 92%, 93%, 94%, 95%, 96%, 97%, 98%, 99%, or 100% of the amino acids or nucleotides of the relevant lengths of the reference sequence. When the amino acid residues or nucleotides at corresponding amino acid positions or nucleotide positions are compared, when a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap.

Suitably, the TRAP protein comprises an amino acid sequence having at least 80%, preferably at least 85%, preferably at least 90%, preferably at least 95%, preferably at least 97% identity or homology to the amino acid sequence of SEQ ID NO: 1. Preferably, the TRAP protein comprises an amino acid sequence having at least at least 85% identity or homology to the amino acid sequence of SEQ ID NO: 1.

Reference herein to “TRAP-cage” refers to an assembled protein complex formed from multiple biomolecules, here multiple TRAP protein rings forming the complex. The TRAP protein rings can be linked together by crosslinkers, herein molecular cross-linkers. “Complex”, “assembly”, “aggregate”, are used alternatively in the description and means a superstructure constructed by the reaction between biomolecules. The amount of the units involved in the complex depends on the nature of the biomolecule. More specifically, it depends on the amount of the biomolecule and the amount of —SH groups present in the biomolecule. “TRAP-cage” and “artificial TRAP-cage” are used interchangeably herein.

TRAP protein is a suitable biomolecule model for the method of the invention. This is likely due to its high intrinsic stability, toroid shape, lack of native cysteine residues (for easier control of the conjugation process) and availability of a residue that can be changed to cysteines with the resulting cysteine being in a suitable chemical and spatial environment suitable for proper bond formation.

Reference herein to “programmable” is intended to convey that the TRAP-cages of the present invention have properties conferred on, or engineered into them that make them prone or susceptible or predisposed to behave in a particular and selected manner on exposure to specific environmental conditions or stimuli.

Reference herein to “open” is synonymous with the TRAP-cage, fracturing, leaking, fragmenting, breaking or generally allowing a cargo to escape from the interior of the cage.

Reference herein to “closed” is synonymous with the TRAP-cage remaining intact, unbreakable, impervious or generally remaining as a whole cage.

Reference herein to “bisfunctional” refers to a molecular crosslinker which has two functional groups, for example herein a molecule with two functional groups, where there is one functional group for each of the cysteine thiol groups to be crosslinked in order to connect TRAP rings in a TRAP-cage. Reference herein to “homobisfunctional” refers to a bisfunctional linker where the two groups are the same.

Preferably, homobisfunctional linkers include bismaleimideohexane (BMH), dithiobismaleimideoethane (DTME), bis-halomethyl benzene and its derivatives, 2-bis-bromomethyl-3-nitrobenzene (BBN), bis-bromoxylene and 1,3-bis-bromomethyl-4,6-dinitro-benzene (BDNB).

“Unit”, “subunit”, “molecule”, “biomolecule”, “monomer” are used alternatively in the description and means one molecule which connects to another molecule for the complex formation.

“Molecular cross-linker” is a molecule that acts to connect units, subunits, molecules, biomolecules or monomers to other examples of the same via formation of one or more chemical bonds. Molecular crosslinkers are not single atoms linkers, which are distinct entities.

Reference herein to a “Reduction resistant/insensitive molecular cross-linker” is reference to a cross-linker which is not cleaved by reduction reaction such as that typically seen when a disulphide bond is cleaved by a reducing agent. These cross-linkers are stable under conditions that would result in breaking of reduction sensitive bonds. These bismaleimideohexane (BMH) and bis-bromoxylenes.

Reference herein to a “Reduction responsive/sensitive molecular cross-linker” is reference to a cross-linker which is cleaved by reduction reaction such as that typically seen when a disulphide bond is cleaved by a reducing agent. These cross-linkers are not stable under conditions that would result in breaking of reduction sensitive bonds. These include dithiobismaleimideoethane (DTME).

Reference herein to a “Photoactivatable molecular cross-linker” is reference to a cross-linker that is photoreactive or sensitive to light, i.e. one that will be cleaved when exposed to light. This light can be UV or other such light of a specific range of wavelengths. These include, 2-bis-bromomethyl-3-nitrobenzene (o-BBN), 2,4-bis-bromomethyl-1-nitrobenzene (m-BBN) and 1,3-bis-bromomethyl-4,6-dinitro-benzene (BDNB).

To achieve a programmable, rather than simply triggerable, disassembly, it has been proposed a protein cage in which building blocks are held together by simple cross-linkers via facile chemistry allowing easy interchangeability (FIG. 1a). Disassembly of such cages would depend on cleavage characteristics of cross-linkers employed. To test this possibility, TRAP (K35C/R64S) has been used and attempted to link TRAP rings with simple bifunctional molecular cross-linkers, either dithiobismaleimideoethane (DTME) or bismaleimideohexane (BMH). They acted as connectors like Au(I) in the gold-mediated assembly (TRAP-cage^{Au(I)}) owing to the thiol-specific reaction at neutral pH. As DTME contains a cleavable disulfide bond but BMH does not, the resulting two cages (TRAP-cage^{DTME} and TRAP-cage^{BMH}) have contrasting disassembly characteristics when exposed to reducing agents (FIG. 1a).

To obtain the covalently cross-linked cages, TRAP (K35C/R64S) was mixed with either DTME or BMH in an aqueous buffer. Size-exclusion chromatography (SEC) of the resulting reaction mixtures showed a substantial peak at the elution volume similar to that of TRAP-cage^{Au(I)}) suggesting successful cage formation with both cross-linkers (FIG. 1). The isolated fractions were further analysed by SEC (FIG. 1c), dynamic light scattering (DLS), and negative-stain transmission electron microscopy (TEM) (FIG. 1d, FIG. 2), yielding results as expected for monodisperse spherical cage structures approximately 25 nm in diameter. Assembly appeared essentially complete within 60 min (FIG. 1b) with a typical yield of around 20% for both cross-linked TRAP-cages. No free cysteines were detected post reaction. Further analysis of the obtained TRAP-cages using size-exclusion chromatography coupled with right and low angle light scattering (SEC-RALS/LALS) indicated the apparent average molecular masses of both particles as 2.2 MDa suggesting a 24-ring arrangement.

The detailed structures of both TRAP-cage^{DTME} and TRAP-cage^{BMH} were determined using cryo-EM single particle reconstruction. Electron density maps at 4.7 Å and 4.9 Å resolution for both types of cages have been obtained. These revealed each structure to be composed of 24 TRAP rings arranged into two chiral forms, similar to that seen for TRAP-cage^{Au(I)} (FIG. 1e). TRAP ring models were refined against the maps producing a good fit. Closer examination at the ring-ring interface found two substantial electron densities bridging two adjacent subunits, which likely corresponds to the bismaleimide cross-linkers (FIG. 1e). The cross-linkers appear to be bent in a horseshoe shape between the cysteine residues of opposing subunits.

Both TRAP-cage^{DTME} and TRAP-cage^{BMH} showed similarly high stability in response to elevated temperatures, chaotropic agents and surfactants. Specifically, they displayed no significant morphology change after 10 minutes incubation at 75° C., pHs in the range 2-11, up to 4 M GndHCl, up to at least 7 M urea and 7% of SDS. However, TRAP-cage^{DTME} readily disassembles upon addition of reducing agents, tris(2-carboxyethyl) phosphine (TCEP) or dithiothreitol (DTT) (FIG. 2a, c). In contrast, TRAP-cage^{BMH} was unaffected (FIG. 2b, c). High stability at high temperatures, large pH ranges and in the presence of high concentrations of cahortopic agents and surfactants was also observed for both TRAP-cage^{DTME} (FIG. 2d-g) and TRAP-cage^{BMH}. DTT-dependent disassembly was further investigated at the single-cage level in real time using high speed atomic force microscopy (HSAFM, FIG. 21). This showed that TRAP-cage^{BMH} was resistant to disassembly in the presence of DTT. In contrast TRAP-Cage^{DTME} under the same conditions readily disassembled with discrete patches of TRAP subunits appearing to “peel off” from the cage surface, eventually leading to the opening of the whole structure approx. 3 min after the first ring detachment (FIG. 2d). Such stepwise disassembly process of TRAP-cage^{DTME} is a marked contrast to TRAP-cage^{Au(I)} which shows a more concerted disassembly on a much shorter time scale.

In order to efficiently track cage stability against various thiol-containing reagents, a spectroscopic method has been employed to develop real-time monitoring of bulk cage disassembly in solution. In this system, two fluorescent proteins, mOrange2 and mCherry, have been encapsulated, serving as a Förster resonance energy transfer (FRET) donor and acceptor, respectively. These fluorescent proteins were genetically fused to the TRAP N-terminus, which faces the cage interior in the assembled structures. In order to avoid steric hinderance during cage formation they were co-produced with unmodified TRAP to form “patchwork” rings.

The resulting TRAP rings were then assembled into cage structures using either Au(I) or DTME (FIG. 3a). After purification using size-exclusion chromatography, isolated particles were analysed by native PAGE (FIG. 3b), and TEM imaging (FIG. 3c), which confirmed guest encapsulation in the lumen of monodisperse spherical cages. The presence of both proteins in the constrained volume of the TRAP-cage lumen should enable efficient FRET (22, 23). Indeed, normalised fluorescence spectra of TRAP-cage^{Au(I)} and TRAP-cage^{DTME} co-packaging both guests showed an approximately 1.5-fold higher signal in mCherry emission at 610 nm, compared to the corresponding control samples containing cages encapsulating only mOrange2 or mCherry and mixed in the solution (FIG. 4a, b). However, the addition of DTT which induces disassembly for both types of cages led to FRET cancellation as observed in the resulting spectra which closely resemble the control samples (FIG. 4a, b). These results indicated efficient energy transfer between encapsulated guests and its cancellation by their release, indicating that the FRET-cages are suitable for monitoring the TRAP-cage disassembly process.

We next measured the disassembly kinetics of both TRAP-cage^{Au(I)} and TRAP-Cage^{DTME} upon the addition of DTT. The change in the fluorescence intensity ratio at 568/610 nm was exploited as an indicator of the time-dependent disassembly process (FIG. 4c). For both cages, increase in the fluorescence reached a plateau approximately 5 min after addition of DTT, indicating complete cage disassembly and guest release. However, the mechanism of this process appeared to be different depending on cage type. In contrast to TRAP-cage Au(I), TRAP-cage^{DTME} displayed sigmoidal disassembly behaviour indicating multiple-steps involved in the guest release procedure. These results well-agreed with concerted versus stepwise disassembly of TRAP-cage^{Au(I)} or TRAP-cage^{DTME}, respectively, observed by HSAFM.

Protein cages able to carry cargo and disassemble in presence of reducing agents have potential for intracellular delivery. Ideal nano-vehicles used for this purpose should remain assembled under extracellular conditions but disassemble upon exposure to intracellular conditions to liberate their cargoes. We assessed this possibility by monitoring cage disassembly kinetics in the presence of cysteine and glutathione employed as model thiol. A time-dependent increase in FRET cancellation was observed for TRAP-cage^{Au(I)} upon addition of cysteine, plateauing after approx. 15 min, indicating complete cages disassembly (FIG. 4d, black circles). In contrast, TRAP-cage^{DTME} containing the same FRET pair showed little fluorescent change, suggesting the cage did not disassemble under these conditions (FIG. 4d, red circles). This stability towards cysteine is due likely to the slow kinetics of disulfide cleavage catalyzed by this free thiol in contrast to a likely much faster ligand exchange for TRAP-cage^{Au(I)}. Nevertheless, TRAP-cage^{DTME} is still able to disassemble by using higher concentrations of free thiol compounds: When 50 mM of reduced glutathione was used disassembly of and cargo release from TRAP-cage^{DTME} was observed though at a significantly slower disassembly rate compared to TRAP-cage^{Au(I)} (FIG. 4e, red and black circles).

According to an aspect, the cages as described herein may be used as medicaments. This could be in a of treating a patient, such as comprising administering a cage as described herein to a patient, or the cages as described herein for use in treating a disease in a patient. This particularly may be a cage designed to carry cargo and disassemble in presence of reducing agents for intracellular delivery. These cages may be administered along with or in the presence of a pharmaceutically acceptable carrier, adjuvant or excipient. The cargo that the cages for use as a medicament or for treating patients will be of benefit to said patient. For example as drug delivery systems (DDS)—for active molecules (especially biological macromolecules such as RNA, DNA, peptides and proteins). They provide advantages ast as biological macromolecules are often easily disrupted or digested by conditions such as those found in vivo. Biological macromolecules are too big to diffuse out of the holes in TRAP, being a large protein, TRAP-cage can sustain significant changes without disrupting overall structure. This means that it can be modified to capture therapeutic cargoes and simultaneously be modified, on the exterior to target therapeutic targets. Programmable linkers can be used which cleave in a desired situation that correlates with arrival at site of action. For example, light could be shone on the target site to cleave open photocleavable TRAP-cages. If TRAP-cages penetrate cells, those held together by reducible linkers will spontaneously open up and release cargo as the cytoplasm of the cell is highly reducing. Cages could also be used in conjunction with vaccines or acting as vaccines, where antigens (i.e. peptides) which are expected to stimulate a T-cell response are captured inside the TRAP-cage and then targeted at to T-cells, followed by triggered opening.

### BRIEF DESCRIPTION OF THE FIGURES

FIG. 1. Molecular cross-link-mediated TRAP-cage formation. a, Schematic representation of the cross-linking reaction with dithiobismaleimideoethane (DTME) or bismaleimideohexane (BMH). TRAP(K35C/R64S) rings, shown on left, with the cysteines represented as circles on the exterior are covalently connected to each other via reaction between cysteines and bismaleimide compounds (line above first arrow with detailed chemical structure below) to form a cage-like structure. Addition of dithiothreitol (DTT) results in disassembly of DTME-mediated cages (TRAP-cages^{DTME}, top) but has no effect on cages assembled with BMH (TRAP-cages^{BMH}, bottom). b, NATIVE-PAGE gel of the TRAP-cages^{DTME} and TRAP-cages^{BMH} formation with black arrow head marking the height of formed cages c, Size-exclusion chromatography profiles of the purified TRAP-cages^{DTME} (light-grey line) and TRAP-cages^{BMH} (grey line). The profile of TRAP-cage^{Au(I)} (black line) is provided as a control. mAu, milliabsorbance units d, Transmission electron microscopy (TEM) images of TRAP-cages^{DTME} (right) and TRAP-cages^{BMH} (left). Scale bars, 50 nm. e, Cryo-electron microscopy density maps of the left-handed (top) and right-handed (bottom) forms of TRAP-cage^{DTME}, refined to 4.7 Å and 4.9 Å resolution, respectively. Inset shows the amplified image at the ring-ring interface with the fitted cross-linker models highlighted for DTME (middle) and BMH (right) shown in side view (top) and top view (bottom).

FIG. 2. Stability of cross-linked TRAP-cages. Redox responsiveness. a,b, Native PAGE analysis of TRAP-cage^{DTME} (a) and TRAP-cage^{BMH} (b) in the presence of DTT and tris(2-carboxyethyl) phosphine (TCEP). TRAP-cage appears as a prominent band running between 1048 and 1236 kDa. C=TRAP-cage^{DTME} (a) and TRAP-cage^{BMH} (b). M=molecular weight marker. c, TEM images showing TRAP-cage^{DTME} after treatment with 0.1 mM (left) and 1 mM (middle) TCEP and TRAP-cage^{BMH} after treatment with 10 mM TCEP (right). Scale bar, 50 nm. d-g stability of TRAP-cage^{DTME}. d, Native PAGE showing Thermal stability of TRAP-cage^{DTME} over indicated incubation times and temperatures. Image below the gel shows TRAP-cage retains its structure after incubation at 95° C. for 10 min., scale bar, 100 nm e, Native PAGE showing effect of pH on stability: Cages are stable at pH 3-11 using native PAGE. Images below gels are TEM images of samples after incubation at the indicated pHs. Scale bar, 100 nm. f, The structure of TRAP-cage^{DTME} as assessed using native PAGE which shows it to be unaffected in the presence of guanidine hydrochloride (GdnHCl), urea and g, SDS over the range tested. TEM image (below) was obtained after incubation of cages with 4 M GndHCl, scale bar, 100 nm. h-k stability of TRAP-cage BMH, As for TRAP-cage^{DTME} results (panels d-g) except TRAP-cage^{BMH} was used. Black arrowheads indicate position of intact TRAP-cage on the gel 0.1, Frames from HSAFM movies showing the effect of 4 mM DTT addition to TRAP-cage^{DTME}. Time after addition of DTT is as indicated.

FIG. 3. Loading TRAP-cages with FRET pairs. a, Schematic representation of TRAP-cage loading with fluorescent proteins. Patchwork TRAP rings fused with either mCherry (black cylinder) or mOrange2 (grey cylinder) at the N-terminus were mixed together with either DTME or triphenylphosphine monosulfate (TPPMS)-Au(I)—Cl. b, Native PAGE showing the fluorescent properties of purified TRAP-cages associated with the fluorescent cargoes. The gel was visualized using InstantBlue protein staining (right) and fluorescence using excitation at 532 nm and emission at 610 nm (left). Note that the exact position of the prominent band corresponding to TRAP-cage running at approx. 1028-1236 kDa varies slightly according to presence/absence of cargo and nature of the crosslinking agent used. c, TEM images of empty (left) TRAP-cages and those filled with fluorescent proteins (right), assembled using either Au(I) (top) or DTME (bottom). Scale bars, 50 nm.

FIG. 4. Guest release. a, b, Normalized emission spectra of TRAP-cages^{Au(I)} (a) and TRAP-cages^{DTME} (b) loaded with both mOrange2 and mCherry upon excitation at 510 nm before and after addition of 10 mM DTT. mOrange2 emission peak at 568 nm, mCherry emission peak at 610 nm. Additional lines indicate spectra of cages loaded only with mOrange2 or mCherry proteins mixed together immediately prior to measurement in the absence or presence of DTT, respectively. c, d, e, Time-dependent disassembly of TRAP-cages^{Au(I)} (black circles) and TRAP-cages^{DTME} (grey circles) after addition of 10 mM DTT (c), 2.5 mM cysteine (Cys) (d) or 50 mM glutathione (GSH) (e). 100% leakage stands for the highest donor intensity upon 10 mM DTT treatment for 10 min after each experiment.

FIG. 5. TRAP cages with different metal linkers. a. Native PAGE analysis for TRAP-cage assembled with Ag(I) or Cd(II); b. TEM image of TRAP-cage^{Au(I)}; c. TEM image of TRAP-cage^{Cd(I)}; d. Native PAGE analysis for TRAP-cage assembled with Co(II) or Zn(II); e. TEM image of TRAP-cage^{Co(II)}; f. TEM image of TRAP-cage^{Zn(II)}; For a. and d. samples were run on 3-12% native Bis-Tris acrylamide gels. Protein bands were visualized by Coomassie Blue staining. For all TEM images—scale bar, 100 nm.

FIG. 6. Native PAGE of templating reaction; M—marker; R—TRAP rings; C—TRAP-cage control; C+T—TRAP-cage+10 mM TCEP showing cage disassembly due to the presence of Au(I); D=TRAP rings+DBX—no cages assembly; DC—TRAP-cage+DBX; DCT—TRAP-cage+DBX+10 mM TCEP—cages are still present suggesting a successful exchange reaction; b. Structure of 1,3-dibromoxylene; c. DLS showing the approx. size of DBX TRAP-cages—24 nm; d. TEM image of purified DBX TRAP-cages after treatment with 10 mM DTT, scale bar, 100 nm; e. cryoEM structures of leavo (left) and dextro (right) structures of DBX crosslinked cages; f. wireframe models of the Au(I) induced cage (left) and DBX cage (right) with zoom on the ring-to-ring connection nature g. Structure of 1,2-bisbromomethyl-3-nitrobenzene (BBN); h. SDS PAGE before and after ‘templating reaction with BBN; M-marker, R-TRAP-rings, C—Au(I) induced TRAP-cage, BBN—TRAP-cage after mixing with BBN—appearance of additional band showing the presence of covalently bound TRAP dimers; i. TEM image of purified BBN TRAP-cages, scale bars, 100 nm (left), 50 nm (right), j. Native PAGE showing the j. dependency of the photocleavage on the presence of different quencher (DTT) concentrations after 10 min UV irradiation, k. different time points after the start of UV irradiation in the presence of 10 mM DTT; for both gels: M—marker, C—TRAP-cage, C+ or CDTT—cross-linked TRAP-cage with reducing agent added to show its resistance towards reducing conditions

FIG. 7. CryoEM density maps showing the structure of TRAP-cage made using TRAP S33C/R64S, resulting in a 20-ring cage. From left to right, view centered on the 4-fold hole; view centered on bowtie hole; perspective view; scale bar—5 nm

### EXAMPLES

Techniques Employed in the Realisation of the Invention

Transmission Electron Microscopy (TEM)

Samples were typically diluted to a final protein concentration of 0.025 mg/ml, centrifuged briefly in a desktop centrifuge and the supernatant applied onto hydrophilized carbon-coated copper grids (STEM Co.), negatively stained with 4% phospotungstic acid, pH 8, and visualized using a JEOL JEM-1230 80 kV instrument.

Native PAGE

Samples were run on 3-12% native Bis-Tris gels following the manufacturer's recommendations (Life Technologies). Samples were mixed with 4× native PAGE sample buffer (200 mM BisTris, pH 7.2, 40% w/v Glycerol, 0.015% w/v Bromophenol Blue). As a qualitative guide to molecular weights of migrated bands, NativeMark unstained protein standard (Life Technologies) was used. Where blue native PAGE was performed, protein bands were visualized according to the manufacturer's protocol (Life Technologies), otherwise InstantBlue™ protein stain (Expedeon) was used.

Protein Expression and Purification

In a typical purification, E. coli BL21(DE3) cells (Novagen) transformed with pET21b plasmid harboring the TRAP (K35C/R64S) gene were grown at 37° C. with shaking in 3 L of LB medium with 100 μg/ml ampicillin until OD₆₀₀=0.6, induced with 0.5 mM IPTG then further shaken for 4 h. Cells were harvested by centrifugation and the pellet kept at −80° C. until use. Cells were lysed by sonication at 4° C. in 50 ml of 50 mM Tris-HCl, pH 7.9, 50 mM NaCl in presence of proteinase inhibitors (Thermo Scientific) and presence or absence of 2 mM DTT, and lysates were centrifuged at 66,063 g for 0.5 h at 4° C. The supernatant fraction was heated at 70° C. for 10 min, cooled to 4° C., and centrifuged again at 66,063 g for 0.5 h at 4° C. The supernatant fraction was purified by ion exchange chromatography on an ÄKTA purifier (GE Healthcare Life Sciences) using 4×5 ml HiTrap QFF columns with binding in 50 mM Tris-HCl, pH 7.9, 0.05 M NaCl, +/−2 mM DTT buffer and eluting with a 0.05-1 M NaCl gradient. Fractions containing TRAP protein were pooled and concentrated using Amicon Ultra 10 kDa MWCO centrifugal filter units (Millipore) and the sample subjected to size exclusion chromatography on a HiLoad 16/60 Superdex 200 column in 50 mM Tris-HCl, pH 7.9, 0.15 M NaCl at room temperature. Protein concentrations were calculated using the BCA protein assay kit (Pierce Biotechnology).

Cages' Stability

Stability of TRAP-cages against chemicals and heat were tested using a similar method to that described previously¹. All agents used for the assays (DTT, TCEP, SDS, Gdn-HCl, and urea) were reconstituted in PBS pH 7.4 and mixed with TRAP-cage samples at room temperature for overnight. Thermal stability check was performed by heating samples at varied temperatures for 10 min. The samples were then subjected to native PAGE. These experiments were repeated twice, each giving uniform results.

Cryo-EM

Preparation of vitreous ice was carried out using 4 μL of protein samples at ˜1 mg/mL in PBS. After blot the samples on EM grids (Quantifoil 1.2/1.3, Cu, 300 mesh), they were plunged frozen in liquid ethane using a FEI Vitrobot with parameters; blot force=0, blot time=4 sec, wait time=0 sec, drain time=0 sec. Micrographs were collected using a FEI TitanKrios cryo-microscope with 300 kV operation and a Falcon III camera at 75 k magnification. 4942 and 10169 micrographs were collected for TRAP-cage^{DTME} and TRAP-cage^{BMH} respectively. All micrographs were motion corrected using MotionCorr2⁶ and CTF estimation was performed using CTFFIND4⁷. Particles were picked and extracted using cryoSPARC v2.12.4⁸ firstly in manual mode (about 4000 particles), followed by automated mode, where initial 2D classes served as a template. Extracted particles were 2D classified again to select best particles for subsequent reconstruction steps. 3D reconstruction was performed by the Heterogenous Refinement protocol using EMD-4443 and EMD-4444 (TRAP-cage^{Au(I)}) as searching models.

### Example 1. TRAP-Cage^{DTME} and TRAP-Cage^{BMH} Preparation

Molecular Cloning

For all cloning steps E. coli NEB 5 alpha strain was used. Plasmid sequences were confirmed by Sanger sequencing method performed by Eurofins. Tetracycline-inducible protein expression vectors were constructed by subcloning gene segment encoding TRAP(K35C) into pACTet_H-mCherry or pACTet_H-mOrange. The gene for TRAP(K35C) was amplified by PCR using pET21b_TRAP-K35C as a template and oligonucleotides, FW_Xhol_TRAP and RV_MluI_TRAP (see Table 3), as primers. The amplified PCR product was directly used as a template for the second PCR which introduced linker gene segment using FW_BsrGl_tev and RV_MluI_TRAP oligonucleotides as primers. The PCR product were cloned into pACTet_H-mCherry or pACTet_H-mOrange via the BsrGl and MluI sites to give pACTet_H-mCherry-TRAP-K35C and pACTet_H-mOrange-tev-TRAP-K35C. Here, only single point mutation K35C, crucial for the subunits linkage, was introduced to the TRAP sequence as previously used R64S was only important to avoid gold nanoparticle binding¹.

TABLE 3
Sequences of oligonucleotides

| Name | Sequence |
|---|---|
| FW_XhoI_TRAP | CTGTACTTCCAGAGCGGCGGTAGCGGCTCGAGCTACACCA |
| | ACTCTGACTTCGTTG [SEQ ID NO: 3] |
| RV_MluI_TRAP | CTCACGCGTTATTTTTTACCTTCAGATTCGATAACAC |
| | [SEQ ID NO: 4] |
| FW_BsrGI_tev | GCTGTACAAGCTTTCTGAAAACCTGTACTTCCAGAGCGGC |
| | [SEQ ID NO: 5] |

Protein Expression and Purification

TRAP proteins were produced using essentially the same protocol as described previously in A. D. Malay, et al., ‘An ultra-stable gold-coordinated protein cage displaying reversible assembly’. Nature 569, 438-442 (2019), which is hereby incorporated by reference but 2 mM DTT was kept in the buffers for the initial purification steps to avoid undesired cysteine oxidation. In order to produce patchwork TRAP rings, E. coli strain BL21(DE3) cells were co-transformed with either pACTet_H-mOrange-TRAPK35C or pACTet_H-mCherry-TRAP-K350 and pET21_TRAP-K350 (See Table 4 in Materials and methods). Cells were grown in 100 ml LB medium supplemented with ampicillin and chloramphenicol at 37° C. until OD₆₀₀=0.5-0.7. At this point, protein expression was induced by addition of 0.2 mM IPTG and 10 ng/ml of tetracycline in case of pACTet_H-mCherry-TRAP-K35C or 30 ng/ml of tetracycline in case of pACTet_H-mOrange-TRAP-K35C, followed by cell culture for 20 hours at 25° C. The cells were then harvested by centrifugation for 10 min at 5,000×g. Cell pellets were stored in −80° C. until purification. They were then resuspended in 40 ml lysis buffer (50 mM sodium phosphate buffer, 600 mM NaCl, 10 mM imidazole, pH 7.4) supplemented with the end of spatula DNase I and lysozyme, 1 tablet of protease inhibitor cocktail and 2 mM DTT and stirred for 30 min at room temperature. Then, the samples were sonicated and clarified by centrifugation at 10,000×g and 4° C. for 20 min. The supernatant was then incubated with 4 ml Ni-NTA resin previously equilibrated in a lysis buffer in a gravity flow column for 20 min. The resin was then washed more than 10 column volumes in lysis buffer containing 20 and 40 mM imidazole. His-tagged proteins were eluted using 5 ml of 50 mM sodium phosphate buffer containing 500 mM imidazole (pH 7.4). Protein samples were then buffer exchanged using Amicon Ultra-15 centrifugal filter unit (50 k molecular weight cut-off (MWCO)) (Merck Millipore) to 2× phosphate buffered saline (PBS) supplied with 5 mM ethylenediaminetetraacetic acid (EDTA), referred to as 2×PBS-E hereafter. The proteins were then subjected to size-exclusion chromatography using a Superdex 200 Increase 10/300 GL column (GE Healthcare) at 0.8 ml/min flow rate. Main peak showing absorption at 548 nm or 587 nm was pooled and concentrated using Amicon Ultra-15 (50 k MWCO). Protein purity was checked by SDS-PAGE and protein concentration was determined by absorbance measured using UV-1900 UV-Vis Spectrophotometer (Shimadzu) using extinction coefficients: ε_{mCherry 587}=72000 M⁻¹ cm⁻¹, ε_{mOrange 548}=58000 M⁻¹ cm^{-1 2}, ε_{TRAP 280}=8250 M⁻¹ cm⁻¹ (http://expasy.orb/tools/protparam.html). Proteins were stored at 4° C. until use.

TABLE 4
Plasmids and amino acid sequences

| Plasmid name | Plasmid | Gene | Amino acid sequence |
|---|---|---|---|
| pET21b_TRAP- | pET21b | TRAP-K35C | MYTNSDFWVIKALEDGVNVIGLTRGADTRFHHSECLD |
| K35C R64S | | R64S | KGEVLIAQFTEHTSAIKVRGKAYIQTSHGVIESEGKK |
| | | | [SEQ ID NO: 6] |
| pET21b_TRAP- | pET21b | TRAP-K35C | MYTNSDFWVIKALEDGVNVIGLTRGADTRFHHSECLD |
| K35C | | | KGEVLIAQFTEHTSAIKVRGKAYIQTRHGVIESEGKK |
| | | | [SEQ ID NO: 7] |
| pACTet_H- | pACYC | H-mOrange- | MHHHHHHGGSSMVSKGEENNMAIIKEFMRFKVRME |
| mOrange- | | TRAP-K35C | GSVNGHEFEIEGEGEGRPYEGFQTAKLKVTKGGPLP |
| TRAP-K35C | | | FAWDILSPHFTYGSKAYVKHPADIPDYFKLSFPEGFK |
| | | | WERVMNYEDGGVVTVTQDSSLQDGEFIYKVKLRGT |
| | | | NFPSDGPVMQKKTMGWEASSERMYPEDGALKGKIK |
| | | | MRLKLKDGGHYTSEVKTTYKAKKPVQLPGAYIVDIKL |
| | | | DITSHNEDYTIVEQYERAEGRHSTGGMDELYKLSENL |
| | | | YFQSGGSGSSYTNSDFWVIKALEDGVNVIGLTRGADT |
| | | | RFHHSECLDKGEVLIAQFTEHTSAIKVRGKAYIQTRH |
| | | | GVIESEGKK |
| | | | [SEQ ID NO: 8] |
| pACTet_H- | pACYC | H-mCherry- | MHHHHHHGGSSMVSKGEEDNMAIIKEFMRFKVHME |
| mCherry-TRAP- | | TRAP-K35C | GSVNGHEFEIEGEGEGRPYEGTQTAKLKVTKGGPLP |
| K35C | | | FAWDILSPQFMYGSKAYVKHPADIPDYLKLSFPEGFK |
| | | | WERVMNFEDGGVVTVTQDSSLQDGEFIYKVKLRGT |
| | | | NFPSDGPVMQKKTMGWEASSERMYPEDGALKGEIK |
| | | | QRLKLKDGGHYDAEVKTTYKAKKPVQLPGAYNVNIK |
| | | | LDITSHNEDYTIVEQYERAEGRHSTGGMDELYKLSEN |
| | | | LYFQSGGSGSSYTNSDFVVIKALEDGVNVIGLTRGAD |
| | | | TRFHHSECLDKGEVLIAQFTEHTSAIKVRGKAYIQTR |
| | | | HGVIESEGKK |
| | | | [SEQ ID NO: 9] |

Free Thiol Concentration Measurement

Free thiol concentrations of either TRAP-cage^{DTME} and TRAP-cage^{BMH} were assessed using 5,5′-dithiobis-(2-nitrobenzoic acid) (DTNB) reagent according to the producer's protocol. Both samples were concentrated to 0.3 mM using Amicon Ultra-4 centrifugal filter unit (100 k MWCO). Absorbance at 412 nm was measured using Spectramax 190 UV/VIS plate reader (Molecular Devices). The concentration of free thiols in the samples was calculated from the molar extinction coefficient of 2-nitro-5-thiobenzoic acid (14150 M⁻¹ cm⁻¹) and was not detectable for TRAP-cage^{DTME} and TRAP-cage^{BMH}.

Cage Assembly and Purification

For cross-linkers-induced cage assembly, TRAP(K35C/R64S) (100-500 μM) in 2×PBS-E was mixed with 5-fold molar excess of either DTME or BMH and stirred at room temperature for 1 hour. Final DMSO concentration in solution was kept no greater than 12.5%. After the reaction, an insoluble fraction, probably caused by low solubility of cross-linkers in aqueous solutions, was removed by centrifugation for 5 min at 12,000×g. Supernatants were then purified by size-exclusion chromatography using a Superose 6 Increase 10/300 GL column (GE Healthcare) at the 0.5 ml/min flow rate on an ÄKTA purifier (GE Healthcare). Fractions containing cross-linked TRAP-cages were pooled and concentrated using Amicon Ultra-4 (100 k MWCO) centrifugal filter units. Typical yield of obtained cross-linked TRAP-cages was approx. 20%. Formation and purification of gold (I)-induced TRAP-cages were performed as previously described¹. Cage formation with fusion proteins were performed using the same protocols as described for both cross-linked and gold (I)-induced cages with additional Ni-NTA purification step before size-exclusion chromatography to purify the sample from only partially assembled cages (His-tagged mCherry and mOrange2, not protected inside the cages, bind to Ni-NTA column). The protein concentration and ratio of encapsulated guests were estimated using absorbance ratio at 280/548 nm or 280/587 nm. Extinction coefficients used for calculations were ε_{mCherry 587}=72000 M⁻¹ cm⁻¹, ε_{mOrange2 548}=58000 M⁻¹ cm⁻¹, ε_{TRAP 280}=8250 M⁻¹ cm⁻¹. Due to spectral overlap between mCherry and mOrange2, to properly calculate the concentrations of both encapsulated guests, mCherry extinction coefficients was also estimated at 548 nm (ε_{mCherry 548}=42538 M⁻¹ cm⁻¹) using absorbance ratio at 548/587 nm of mCherry without fusion to TRAP. Likewise, extinction coefficient of mCherry and mOrange2 at 280 nm were experimentally determined as ε_{mCherry 280}=56744 M⁻¹ cm⁻¹ and ε_{mOrange2 280}=52200 M⁻¹ cm⁻¹ respectively.

### Example 2. Confirmation of TRAP-Cage Structure Using Cryo-EM

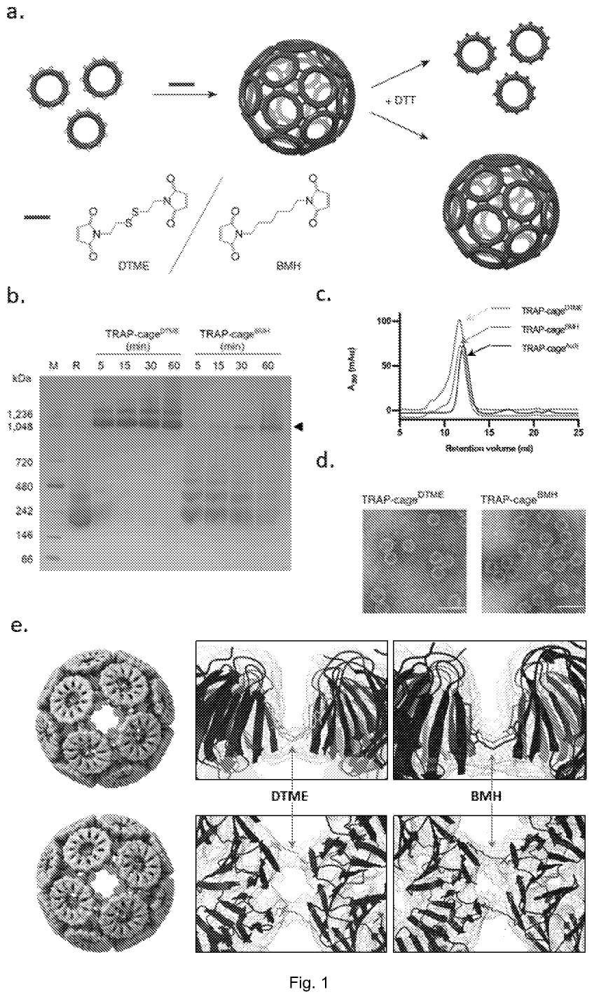

The detailed structures of both TRAP-cage^{DTME} and TRAP-cage^{BMH} were determined using cryo-EM single particle reconstruction. It has been obtained electron density maps at 4.7 Å and 4.9 Å resolution for both types of cages. These revealed each structure to be composed of 24 TRAP rings arranged into two chiral forms, similar to that seen for TRAP-cage^{Au(I)}. TRAP ring models were refined against the maps producing a good fit. Closer examination at the ring-ring interface found two substantial electron densities bridging two adjacent subunits, which likely corresponds to the bismaleimide cross-linkers. The cross-linkers appear to be bent in a horseshoe shape between the cysteine residues of opposing subunits (FIG. 1).

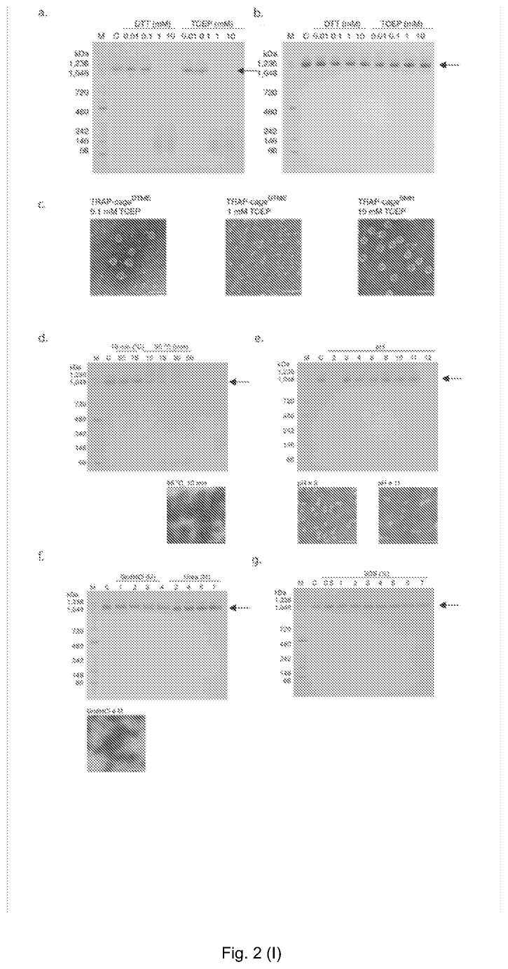

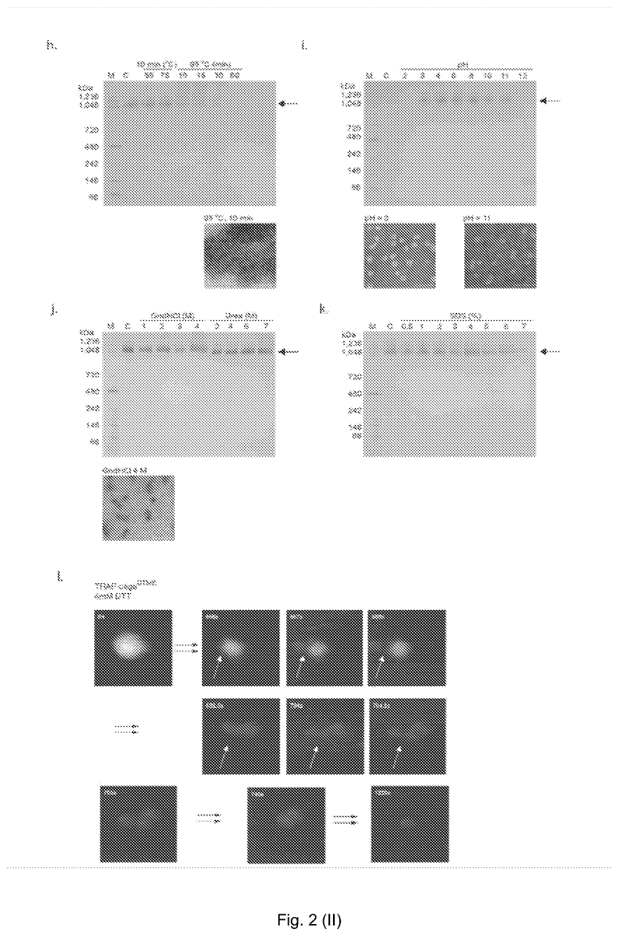

### Example 3. Specific Cleavage Characteristic of the Molecular Cross-Linkers Used in TRAP-Cage (FIG. 2-4)

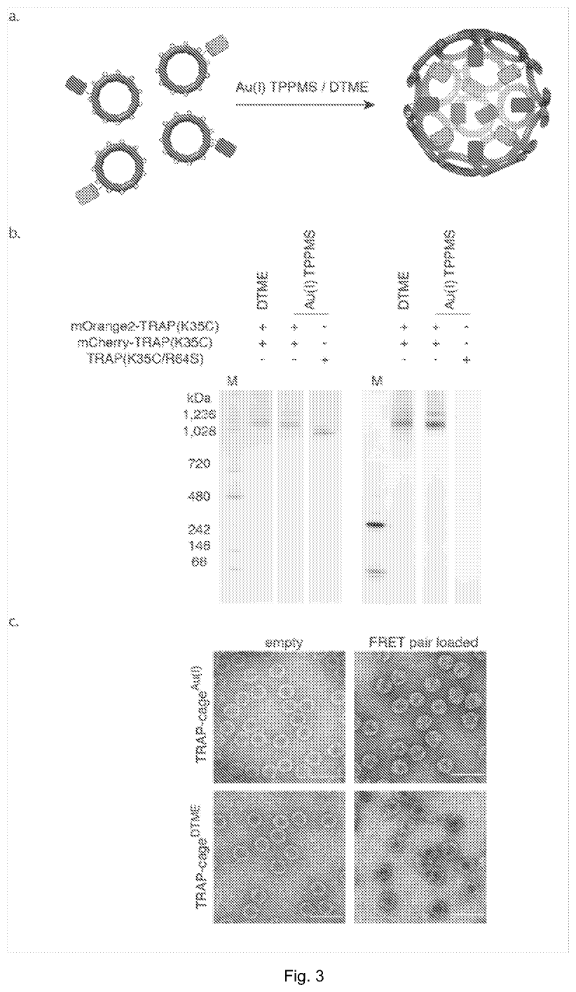

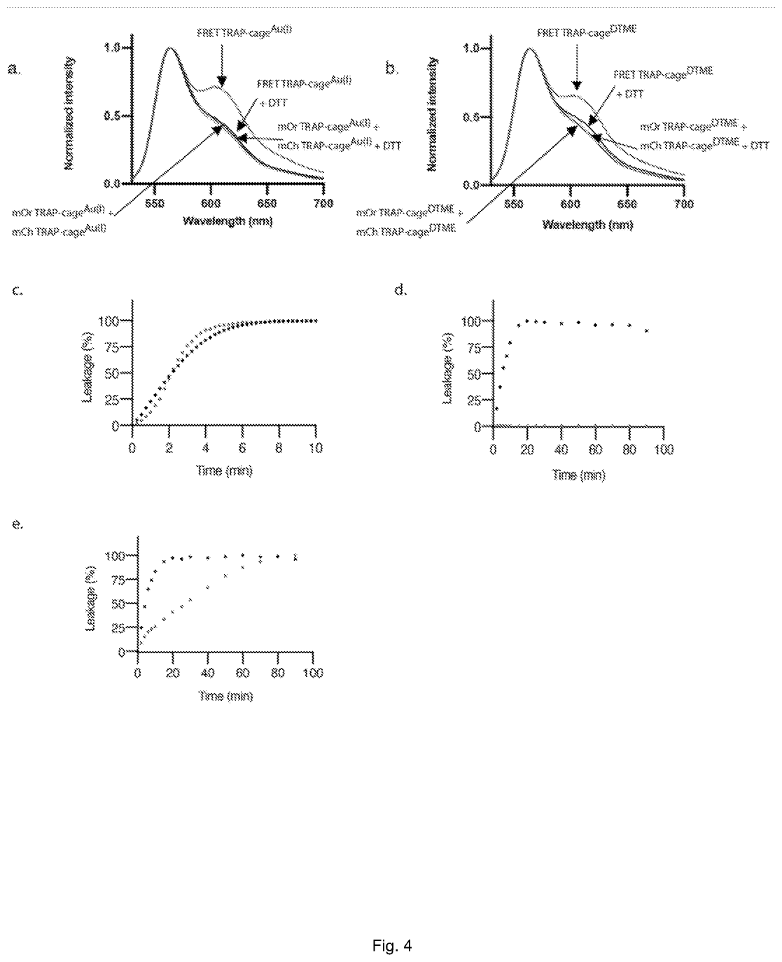

Both TRAP-cage^{DTME} and TRAP-cage^{BMH} showed similarly high stability in response to elevated temperatures, chaotropic agents and surfactants. Specifically, they displayed no significant morphology change after 10 minutes incubation at 75° C., pHs in the range 2-11, up to 4 M GndHCl, up to at least 7 M urea and 7% of SDS. However, TRAP-cage^{DTME} readily disassembles upon addition of reducing agents, tris(2-carboxyethyl) phosphine (TCEP) or dithiothreitol (DTT). In contrast, TRAP-cage^{BMH} was unaffected. DTT-dependent disassembly was further investigated at the single-cage level in real time using high speed atomic force microscopy (HSAFM). This showed that TRAP-cage^{BMH} was resistant to disassembly in the presence of DTT. In contrast TRAP-cage^{DTME} under the same conditions readily disassembled with discrete patches of TRAP subunits appearing to “peel off” from the cage surface, eventually leading to the opening of the whole structure approx. 3 min after the first ring detachment. Such stepwise disassembly process of TRAP-cage^{DTME} is a marked contrast to TRAP-cage^{Au(I)} which shows a more concerted disassembly on a much shorter time scale. Assembly of a TRAP-cage^{DTME} carrying a FRET protein pan crog (FIG. 3) also allowed the disassembly in presence reducing agent to be characterised kinetically (FIG. 4).

### Example 4. Cage Assembly with Different Metals

Protein Expression and Purification

TRAP(K35C/R64S) protein was expressed and purified as described previously.

TRAP(S33H/K35C) and TRAP(S33H/K35H) proteins were expressed and purified according to the same protocol as TRAP(K35C/R64S), but all buffers had pH 8.5.

Protein concentration was determined by measuring absorbance at 280 nm.

Cage Assembly with Different Metals

Formation of TRAP-cages was carried out by mixing purified TRAP variants (final concentration 0.1 mM of monomeric subunits) with salt of relevant metal in a TRAP monomer: metal ion ratio between 4:1-2:1 in suitable buffer: AgNO₃ in 50 mM Tris, pH 7.9, 0.15 M NaNO₃; Cd(NO₃)₂ in 50 mM Tris, pH 7.9, 0.15 M NaCl; CoCl₂ or ZnCl₂ in 50 mM HEPES, pH 7.9, 0.15 M NaCl. Reactions were typically incubated for 3 days at room temperature. Formation of TRAP-cage was confirmed using native PAGE and TEM. Any precipitated material was removed by centrifugation at 12,045 g for 5 min.

Gold-Driven TRAP-Cage [TRAP(K35C/R64S)+Au(I)]

A double mutant of the tryptophan RNA-binding attenuation protein TRAP(K35C/R64S) can assemble into a hollow spherical structure by reaction with monovalent gold ions. Cryo-EM single particle reconstruction revealed that the resulting 22 nm cage is composed of 24 ring-shape undecameric subunits via linear sulfur-Au(I)-sulfur crosslinking between opposing cysteines.

Silver and Cadmium-Driven TRAP-Cages [TRAP(K35C/R64S)+Ag(I) or Cd(II)]

Cage formation can be promoted by other metals than Au(I), namely Hg(II), Ag(I), Cd(II) suggesting that metal-driven cage formation requires water-stable, d10 metal ions with preferred two-ligand linear geometry.

Ag(I)-TRAP-cage is formed and remains stable only in the absence of chloride ions.

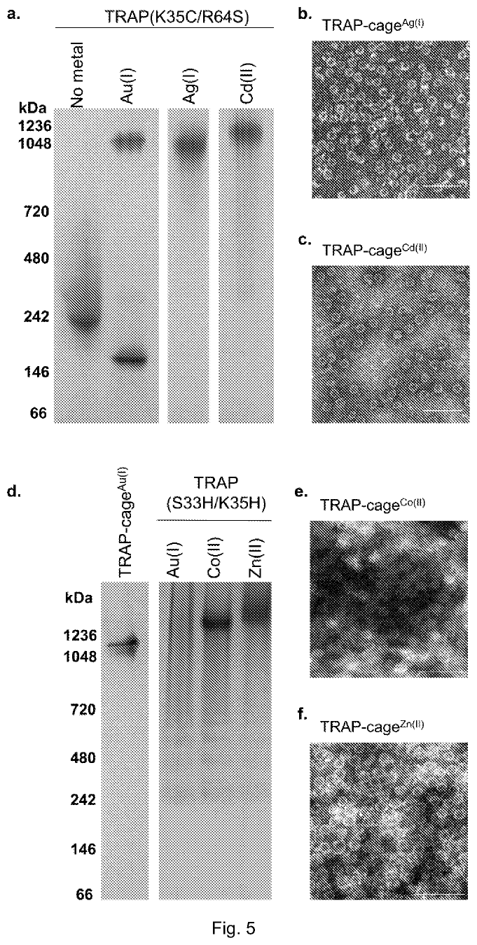

TRAP(K35C/R64S) cages made by addition of Ag(I) or Cd(II) showed the bands on native PAGE with mobility similar to Au(I)-mediated TRAP-cage (FIG. 5a). Cage formation was further confirmed by negative-stain transmission electron microscopy (TEM), showing spherical hollow structures with a diameter 22-24 nm (FIGS. 5b and 5c). These results suggested that silver and cadmium-driven cages likely forms structures with morphology similar to Au(I)-TRAP-cage.

Cobalt and Zinc-Driven TRAP-Cages [TRAP(S33H/K35C) or TRAP(S33H/K35H)+Co(II) or Zn(II)]

The TRAP metal-binding site has been reengineered to target metal ions with preference for tetrahedral coordination. Based on the crystal structure, a pair of histidines or cysteine and histidine were introduced at i and i+2 positions of the β-sheet motif around the rim of the TRAP ring, yielding TRAP(S33H/K35C) and TRAP(S33H/K35H), so that individual monomer unit provides two ligands to coordinate divalent metals. These variants assembled into cage structures upon addition of Zn(II) and Co(II).

Native electrophoresis revealed that TRAP(S33H/K35H) with both Zn(II) and Co(II) migrate similarly as Au(I)-mediated TRAP-cage (FIG. 5d). Formation of cage with a diameter around 22 nm was confirmed by negative-stain transmission electron microscopy (TEM), suggesting resemblance of this structure to Au(I)-TRAP-cage (FIGS. 5e and 5f).

### Example 5. TRAP-Cages Assembled with a Photocleavable Cross-Linker

Materials and Methods:

Gold-induced TRAP-cages were prepared as described previously (Malay et al. Nature, 2019, which is hereby incorporated by reference). 1,3-dibromoxylene and 1,3-bisbromomethyl-4-nitrobenzene were purchased from a commercial vender and dissolved in N, N-dimethyl formamide (DMF). 2 molar excess (to TRAP monomer) of either of cross-linkers were mixed with freshly purified gold-induced TRAP-cage in 50 mM sodium phosphate buffer, pH 7.4 containing 5 mM EDTA while stirring at room temperature for 1 hour. 10 mM dithiothreitol (DTT) was then added to the reaction to capture Au(I). The sample was then purified by size exclusion chromatography using a Superose 6 Increase 10/300 GL column (GE Healthcare).

Photoinduced disassembly of 1,3-bisbromomethyl-4-nitro-benzene TRAP-cage was tested by exposing the samples for varied time with 365-nm wavelength light in the presence of 10 mM dithiothreitol (DTT) to quench the free radical species. The cage morphology and the crosslinker-cleavage process was monitored using dynamic light scattering (DLS) on a Zetasizer (Malvern), SDS, native PAGE and TEM.

Results

In the first attempt, we tried similar method of cages' assembly as we did for BMH/DTME cross-linkers (simple mixing of TRAP rings with the excess of cross-linker) without any success, probably due to the difficulties in the controlling the right orientation of the rings in the structure.

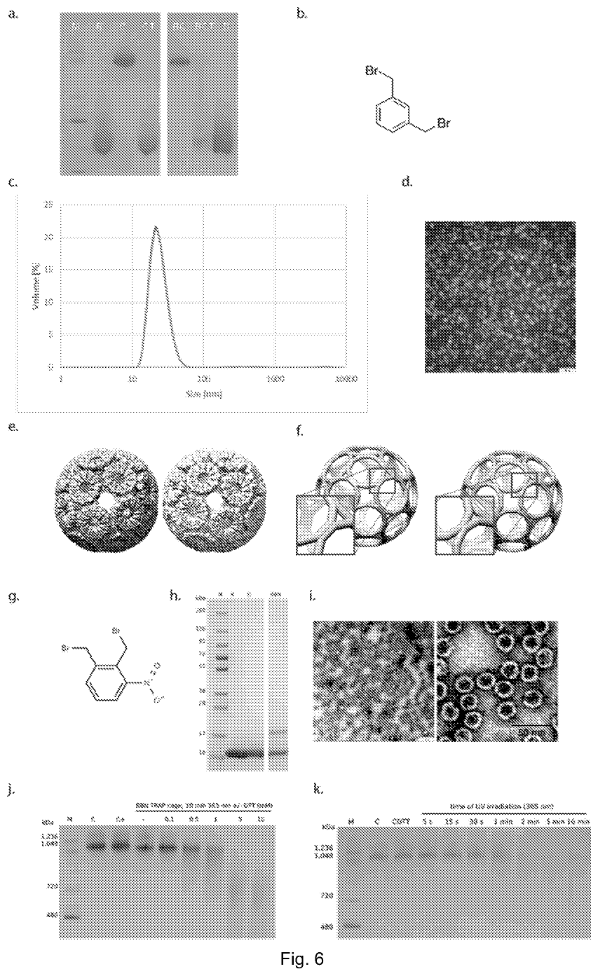

We found a novel method to overcome this problem which uses previously assembled Au(I)-induced TRAP-cages instead of just rings, enabling dibromo-cross-linker to exchange gold atoms without changing orientation of the rings in the cage, which we called ‘templating reaction’. We further used the 1,3-dibromoxylene (DBX) cross-linker (FIG. 6b) as a basic one for optimization of this method. Gold-induced TRAP-cages which possess Au(I) as a linker between rings are prone to disassembly in the presence of reducing conditions (FIG. 6a lane CT). We used this property to check if DBX was built into the structure of TRAP-cage as the result of ‘templating reaction’ due to not having disassembly properties in the same conditions. Indeed, after mixing DBX with Au(I) induced TRAP-cages and further purification, TRAP-cages became resistant to disassembly in reducing conditions proving the presence of a different type of linking between the rings. We further characterized the structure of obtained DBX TRAP-cage by DLS method (FIG. 6c) which indicated the size of DBX TRAP-cages to be approx. 24 nm with high monodispersity. DBX TRAP-cages are 2 nm larger than Au(I) induced TRAP-cages also suggesting the presence of cross-linker in the structure widening their size with the maintenance of the cage-like structure which could be observed on TEM (FIG. 6d). Finally, we solved the structure of DBX TRAP-cages which proved the presence of DBX cross-linker between the rings. CryoEM structures (FIG. 6e) of the resulting cages revealed other interesting features of the assembly. Indeed, like in the case of Au(I) induced cages, we were able to distinguish two chiral forms (leavo and dextro) of the cage, which was not a surprise, because of chiral properties of the gold induced cages. The striking difference between template cage (gold-induced) and the resulting one (DBX-containing) is the number of the connections between the TRAP rings. In case of Au(I) induced cages there were 120 connections identified (—S—Au—S— bridges) but in case of DBX-cage the number of connections drops down to half of that number. 60 linker molecules and same overall geometry (based on snub cube) forces slightly different TRAP rings orientation. Wireframe models of Au(I) induced cage and DBX cages show the difference between TRAP rings orientation and transition from edge-to-edge (Au(I)) to vertex-to-vertex (DBX) (FIG. 6f).

Obtaining the successful results with the basic dibromo-crosslinking of TRAP-cages we decided to change DBX for photolabile cross-linker, very similar in structure, but which, due to the presence of nitro group, can be cleaved after UV (365 nm) light irradiation-1,2-dibromo-3-nitro-benzene (FIG. 6g). We optimized the conditions of ‘templating reaction’ with BBN cross-linker which turned out to be identical to previously used DBX. SDS PAGE showed a clear appearance of TRAP dimers after ‘templating reaction’ proving the presence of covalent bonds (FIG. 6h) suggesting that BBN cross-linker is built into the TRAP-cage structure in the similar manner as previously used DBX. TEM confirmed the presence of monodisperse TRAP-cages with the diameter approx. 24 nm (FIG. 6i).

To further investigate the potential of obtained photolabile TRAP-cages we tested their ability for disassembly under UV light. Such reactions depend on the presence of quenchers which do not allow for the reaction to be reversible. We tested different concentrations of quencher (DTT) and showed such dependency also in the case of BBN TRAP-cages. BBN TRAP-cages were successfully disassembled under UV light and in the presence of 10 mM DTT (FIG. 6j). We also tested how much time it takes to fully disassemble BBN TRAP-cages. Native PAGE showed the gradual disappearance of the cage band in time and the full disassembly was estimated to happen in approx. 2 min from the start of UV irradiation (FIG. 6k).

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### Example 6. A TRAP-Cage Made from Twenty Rings

Materials and Methods

TRAP protein was expressed as described above except the expression plasmid encoded for a TRAP protein having the mutation S33C instead of K35C (with mutation R64S being also). Incubation with a source of Au(I) was similar to as described above with, additionally. Subsequent Purification of the resulting formed TRAP-cages was similar to as described above. Determination of the structure of the resulting TRAP-cage was carried out using CryoEM similar to as described above.

Results

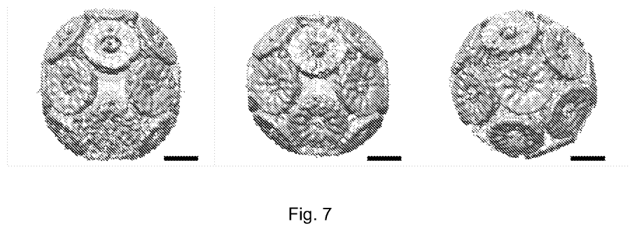

Structural analyses (FIG. 7) of the assembled cage revealed that it is composed of 20 TRAPS33C/R64S rings that are connected with bridging densities reminiscent of those seen in the case of cages seen when using the TRAPK35C mutant). This gives confidence that, despite the poorer resolution in this case, the connections between adjacent rings are the same gold staples as seen in the previous TRAP-cage, with Au(I) ion acting as a bridge between two opposing Cys residues.

1. An artificial TRAP-cage comprising a selected number of TRAP rings which are held in place by molecular cross-linkers, wherein the cross-linkers are selected for their specific characteristics whereby the cages are programmable to be opened or remain closed on demand, under specific conditions.

2. The cage of claim 1, wherein the specific cleavage characteristic of the molecular cross-linker is selected from the group comprising:
(i) a reduction resistant/insensitive molecular cross-linker, whereby the cage remains closed under reducing conditions;
(ii) a reduction responsive/sensitive molecular cross-linker, whereby the cage opens under reducing conditions; and
(iii) a photoactivatable molecular cross-linker whereby the cage opens upon exposure to light.

3. The cage according to either claim 1 or 2 wherein the cross-linker is a homobisfunctional molecular moiety and its derivatives.

4. The cage of any one of claims 1 to 3, wherein the cage is resistant or insensitive to reducing conditions.

5. The cage of claim 4, wherein the cross-linker is a bismaleimideohexane (BMH) or a bis-bromoxylene.

6. The cage of any one of claims 1 to 3, wherein the cage is responsive or sensitive to reducing conditions.

7. The cage of claim 6, wherein the cross-linker is dithiobismaleimideoethane (DTME).

8. The cage of any one of claims 1 to 3, wherein the cage is photoactivatable.

9. The cage of claim 8, wherein the cross-linker is bis-halomethyl benzene and its derivatives including 1,2-bis-bromomethyl-3-nitrobenzene (o-BBN), 2,4-bis-bromomethyl-1-nitrobenzene (m-BBN) and 1,3-bis-bromomethyl-4,6-dinitro-benzene (BDNB).

10. The cage according to either claim 8 or 9 wherein the cross-linker is photolabile by exposure to UV light.

11. The cage according to any preceding claim wherein the number of TRAP rings in the TRAP-cage is between 6 to 60.

12. The cage according to claim 11 wherein the number of TRAP rings in the TRAP-cage is 12, 20 or 24, preferably 24.

13. The cage according to any preceding claim which comprises a mixture of different programmable cross-linkers.

14. The cage according to any preceding claim that encapsulates a cargo that can be programmed to deliver said cargo in a specifically timed and desired location.

15. The cage according to any preceding claim, wherein the artificial TRAP-cage protein is modified to comprise any one or more of the following mutations selected from the group comprising K35C, R64S and K35C/R64S.

16. The cage according to any preceding claim, wherein the cage is stable in elevated temperatures, stable in a non-neutral pH and/or stable in chaotropic agents.

17. An artificial TRAP-cage comprising a selected number of TRAP rings which are held in place by at least one metal cross-linker, wherein the metal is selected from the group comprising Ag(I), Cd(II), Zn(II) and Co(II).

18. The cage according to claim 17, wherein the artificial TRAP-cage protein is modified to comprise any one or more of the following mutations selected from the group comprising K35C, K35H, R64S, K35C/R64S, K35H/R64S, S33C, S33H, S33C/R64S, S33H/R64S, S33C/K35H S33H/K35H, S33C/K35C and S33H/K35C.

19. The cage according to claim 17 or claim 18, wherein the cross-linker comprises cadmium and preferably the artificial TRAP-cage protein is modified to comprise a K35C/R64S mutation.

20. The cage according to claim 17 or claim 18, wherein the cross-linker comprises silver and the artificial TRAP-cage protein is modified to comprise a K35C/R64S mutation.

21. The cage according to claim 17 or claim 18, wherein the cross-linker comprises cobalt and the artificial TRAP-cage protein is modified to comprise a S33H/K35C or a S33H/K35H mutation.

22. The cage according to claim 17 or claim 18, wherein the cross-linker comprises zinc and the artificial TRAP-cage protein is modified to comprise a S33H/K35C or a S33H/K35H mutation.

23. The cage according to any one of claims 17 to 22, wherein the number of TRAP rings in the TRAP-cage is between 6 to 60.

24. The cage according to claim 23, wherein the number of TRAP rings in the TRAP-cage is 12, 20 or 24, preferably 24.

25. The cage according to any one of claims 17 to 24, which comprises a mixture of different programmable cross-linkers.

26. The cage according to any one of claims 17 to 25, which encapsulates a cargo that can be programmed to deliver said cargo in a specifically timed and desired location.

27. The cage according to any preceding claim that is approximately spherical in shape.

28. Use of the cage according to any preceding claim in delivery of a cargo in a controlled period and to a desired location.

29. Use of the cage according to any preceding claim as a medicament.

30. A method of treating a patient, comprising administering a cage according to any one of claims 1 to 27 to said patient.

31. The cage according to any one of claims 1 to 27 for use in treating a disease in a patient.

32. Use of any one or more of the group comprising a homobisfunctional molecular moiety, a bis-halomethyl benzene and its derivatives, as a programmable cross-linker in the construction of a programmable TRAP-cage.

33. Use of any one or more of the metals Ag(I), Cd(II), Zn(II) and Co(II) and their derivates as a cross-linker in the construction of a TRAP-cage.

34. A method of preparing an artificial TRAP-cage, the method comprising:
(i) obtaining TRAP ring units by expression of the TRAP ring units in a suitable expression system and purification of the said units from the expression system;
(ii) conjugation of the TRAP ring units via at least one free thiol linkage with a programmable molecular cross-linker, wherein the cross-linker is selected for its specific characteristics;
(iii) formation of the TRAP-cage; and
(iv) purification and isolation of the TRAP-cages.

35. The method of claim 34, wherein the programmable cross-linker is selected from the group comprising:
(i) a reduction resistant/insensitive linker, whereby the cage remains closed under reducing conditions;
(ii) a reduction responsive/sensitive linker, whereby the cage opens under reducing conditions; and
(iii) a photoactivatable linker whereby the cage opens upon exposure to light.

36. The method according claim 34 or 35 wherein step (ii) comprises conjugation with a mixture of different programmable cross-linkers.

37. A TRAP cage produced by the method of any one of claims 34 to 36.

38. A method of preparing an artificial TRAP-cage, the method comprising:
(i) obtaining TRAP ring units by expression of the TRAP ring units in a suitable expression system and purification of the said units from the expression system;
(ii) conjugation of the TRAP ring units via at least one free thiol linkage with a metal cross-linker, wherein the cross-linker is selected for its specific characteristics;
(iii) formation of the TRAP-cage; and
(iv) purification and isolation of the TRAP-cages;
wherein the metal is selected from the group comprising Ag(I), Cd(II), Zn(II) and Co(II).

39. A TRAP cage produced by the method of claim 38.

40. An artificial TRAP-cage comprising a protein modified to comprise any one or more of the following mutations selected from the group comprising K35C, K35H, R64S, K35C/R64S, K35H/R64S, S33C, S33H, S33C/R64S, S33H/R64S, S33C/K35H S33H/K35H, S33C/K35C and S33H/K35C.
